SEQUENCE LISTING (59 sequences)

SEQ ID NO: 1 - RNA, 20 nt, synthetic construct
  source 1..20: mol_type = other RNA; note = crRNA
agatgaggacgacatcccgc

SEQ ID NO: 2 - RNA, 20 nt, synthetic construct
  source 1..20: mol_type = other RNA; note = crRNA
tgatcggcattgccttcacg

SEQ ID NO: 3 - RNA, 20 nt, synthetic construct
  source 1..20: mol_type = other RNA; note = crRNA
ccgcgttatgtcctgagcat

SEQ ID NO: 4 - DNA, 20 bp, synthetic construct
  source 1..20: mol_type = other DNA; note = Primer DDX41 Exon9-Forward
atcatctgcccctcggtaag

SEQ ID NO: 5 - DNA, 20 bp, synthetic construct
  source 1..20: mol_type = other DNA; note = Primer DDX41 Exon9-Reverse
ccccaactaacctccccatt

SEQ ID NO: 6 - DNA, 20 bp, synthetic construct
  source 1..20: mol_type = other DNA; note = Primer DDX41 Exon10-Forward
atacatagggcaggtggtgg

SEQ ID NO: 7 - DNA, 20 bp, synthetic construct
  source 1..20: mol_type = other DNA; note = Primer DDX41 Exon10-Reverse
agctgagcaactgagacaca

SEQ ID NO: 8 - DNA, 20 bp, synthetic construct
  source 1..20: mol_type = other DNA; note = Primer Sdc1-exon3_4-Forward
gaggatggaactgccaatca

SEQ ID NO: 9 - DNA, 22 bp, synthetic construct
  source 1..22: mol_type = other DNA; note = Primer Sdc1-exon3_4-Reverse
cccagatgtttcaaaggtgaag

SEQ ID NO: 10 - DNA, 19 bp, synthetic construct
  source 1..19: mol_type = other DNA; note = Primer Sdc1-exon4_5-Forward
ggtgcttctcagagccttt

SEQ ID NO: 11 - DNA, 19 bp, synthetic construct
  source 1..19: mol_type = other DNA; note = Primer Sdc1-exon4_5-Reverse
aggcacacagcaaagatga

SEQ ID NO: 12 - DNA, 17 bp, synthetic construct
  source 1..17: mol_type = other DNA; note = Primer Clk3-exon4_5-Forward
ggcgattggctccaaga

SEQ ID NO: 13 - DNA, 17 bp, synthetic construct
  source 1..17: mol_type = other DNA; note = Primer Clk3-exon4_5-Reverse
agcactccaccaccttg

SEQ ID NO: 14 - DNA, 20 bp, synthetic construct
  source 1..20: mol_type = other DNA; note = Primer Clk3-exon9_10-Forward
ccatgaacatcacaccacca

SEQ ID NO: 15 - DNA, 22 bp, synthetic construct
  source 1..22: mol_type = other DNA; note = Primer Clk3-exon9_10-Reverse
ctcaaagagaatgcagccgata

SEQ ID NO: 16 - DNA, 22 bp, synthetic construct
  source 1..22: mol_type = other DNA; note = Primer Fam133b-exon6_7-Forward
tctgattcttccagcagttctt

SEQ ID NO: 17 - DNA, 19 bp, synthetic construct
  source 1..19: mol_type = other DNA; note = Primer Fam133b-exon6_7-Reverse
aggagacttatggcaacgg

SEQ ID NO: 18 - DNA, 20 bp, synthetic construct
  source 1..20: mol_type = other DNA; note = Primer Fam133b-exon7_8-Forward
cgttgccataagtctcctga

SEQ ID NO: 19 - DNA, 24 bp, synthetic construct
  source 1..24: mol_type = other DNA; note = Primer Fam133b-exon7_8-Reverse
tttctctttcagttacatccttgg

SEQ ID NO: 20 - DNA, 22 bp, synthetic construct
  source 1..22: mol_type = other DNA; note = Primer Gas5-exon4_6-Forward
gtcaggaagctggataacagag

SEQ ID NO: 21 - DNA, 20 bp, synthetic construct
  source 1..20: mol_type = other DNA; note = Primer Gas5-exon4_6-Reverse
agcctcaaactccaccattt

SEQ ID NO: 22 - DNA, 21 bp, synthetic construct
  source 1..21: mol_type = other DNA; note = Primer Gas5-exon6_8-Forward
ggtggagtttgaggctggata

SEQ ID NO: 23 - DNA, 17 bp, synthetic construct
  source 1..17: mol_type = other DNA; note = Primer Gas5-exon6_8-Reverse
ccaagcaagccagccaa

SEQ ID NO: 24 - DNA, 17 bp, synthetic construct
  source 1..17: mol_type = other DNA; note = Primer Clk3-ei1-Forward
tgggagacggtgagtgt

SEQ ID NO: 25 - DNA, 21 bp, synthetic construct
  source 1..21: mol_type = other DNA; note = Primer Clk3-ei1-Reverse
gaaataggtccaagccgagac

SEQ ID NO: 26 - DNA, 17 bp, synthetic construct
  source 1..17: mol_type = other DNA; note = Primer Clk3-ei2-Forward
catcccgaagggagcct

SEQ ID NO: 27 - DNA, 19 bp, synthetic construct
  source 1..19: mol_type = other DNA; note = Primer Clk3-ei2-Reverse
agagctctgggccattcta

SEQ ID NO: 28 - DNA, 21 bp, synthetic construct
  source 1..21: mol_type = other DNA; note = Primer Clk3-ei3-Forward
gtaccaggtcttgtagcagtg

SEQ ID NO: 29 - DNA, 22 bp, synthetic construct
  source 1..22: mol_type = other DNA; note = Primer Clk3-ei3-Reverse
gagaaagaggtgaggcctttag

SEQ ID NO: 30 - DNA, 18 bp, synthetic construct
  source 1..18: mol_type = other DNA; note = Primer Clk3-ei3-2Forward
tcacaaacgccgtaccag

SEQ ID NO: 31 - DNA, 21 bp, synthetic construct
  source 1..21: mol_type = other DNA; note = Primer Clk3-ei3-2Reverse
gagaagcgagaaagaggtgag

SEQ ID NO: 32 - DNA, 21 bp, synthetic construct
  source 1..21: mol_type = other DNA; note = Primer Clk3-ie3-Forward
acctcaatctgtcaatcggaa

SEQ ID NO: 33 - DNA, 22 bp, synthetic construct
  source 1..22: mol_type = other DNA; note = Primer Clk3-ie3-Reverse
ctccttgtcatcttccacactc

SEQ ID NO: 34 - DNA, 18 bp, synthetic construct
  source 1..18: mol_type = other DNA; note = Primer Clk3-ie3-2Forward
attggtcggatggcgttt

SEQ ID NO: 35 - DNA, 19 bp, synthetic construct
  source 1..19: mol_type = other DNA; note = Primer Clk3-ie3-2Reverse
ggccctccttgtcatcttc

SEQ ID NO: 36 - DNA, 17 bp, synthetic construct
  source 1..17: mol_type = other DNA; note = Primer Clk3-ei4-Forward
tgccggatcggcgattg

SEQ ID NO: 37 - DNA, 25 bp, synthetic construct
  source 1..25: mol_type = other DNA; note = Primer Clk3-ei4-Reverse
gggtagaaagtagagaggtaaagtt

SEQ ID NO: 38 - DNA, 18 bp, synthetic construct
  source 1..18: mol_type = other DNA; note = Primer Clk3-ei5-Forward
gtggagtgcttggaccat

SEQ ID NO: 39 - DNA, 19 bp, synthetic construct
  source 1..19: mol_type = other DNA; note = Primer Clk3-ei5-Reverse
acccaacacatccagcaat

SEQ ID NO: 40 - DNA, 22 bp, synthetic construct
  source 1..22: mol_type = other DNA; note = Primer Clk3-ei6-Forward
atcaaggagaaagacaaggaaa

SEQ ID NO: 41 - DNA, 20 bp, synthetic construct
  source 1..20: mol_type = other DNA; note = Primer Clk3-ei6-Reverse
gcagctggaactagagaatg

SEQ ID NO: 42 - DNA, 20 bp, synthetic construct
  source 1..20: mol_type = other DNA; note = Primer Clk3-ei7-Forward
gctctgtcatgcccttagat

SEQ ID NO: 43 - DNA, 22 bp, synthetic construct
  source 1..22: mol_type = other DNA; note = Primer Clk3-ei7-Reverse
ggcctgtaagaccttgtagaat

SEQ ID NO: 44 - DNA, 24 bp, synthetic construct
  source 1..24: mol_type = other DNA; note = Primer Clk3-ei8-Forward
ccagagaacatcttgtttgtgaat

SEQ ID NO: 45 - DNA, 18 bp, synthetic construct
  source 1..18: mol_type = other DNA; note = Primer Clk3-ei8-Reverse
cttagagcagggccacac

SEQ ID NO: 46 - DNA, 17 bp, synthetic construct
  source 1..17: mol_type = other DNA; note = Primer Clk3-ei9-Forward
actaccgcccacctgag

SEQ ID NO: 47 - DNA, 25 bp, synthetic construct
  source 1..25: mol_type = other DNA; note = Primer Clk3-ei9-Reverse
tgataactgccagatgtaaacaatg

SEQ ID NO: 48 - DNA, 22 bp, synthetic construct
  source 1..22: mol_type = other DNA; note = Primer Clk3-intron3exon5-Forward (qPCR, PCR)
acctcaatctgtcaatcggaac

SEQ ID NO: 49 - DNA, 17 bp, synthetic construct
  source 1..17: mol_type = other DNA; note = Primer Clk3-intron3exon5-Reverse (qPCR, PCR)
agcactccaccaccttg

SEQ ID NO: 50 - DNA, 19 bp, synthetic construct
  source 1..19: mol_type = other DNA; note = Primer Ybx1-202-Forward
tgcaggagagcaaggtaga

SEQ ID NO: 51 - DNA, 23 bp, synthetic construct
  source 1..23: mol_type = other DNA; note = Primer Ybx1-202-Reverse
ctgaaatgaatgcttcggaatcg

SEQ ID NO: 52 - DNA, 19 bp, synthetic construct
  source 1..19: mol_type = other DNA; note = Primer Ythdf2-202-Forward
tcttctcggacgacttgct

SEQ ID NO: 53 - DNA, 22 bp, synthetic construct
  source 1..22: mol_type = other DNA; note = Primer Ythdf2-202-Reverse
gggctcaagtaaggttcgaaat

SEQ ID NO: 54 - DNA, 20 bp, synthetic construct
  source 1..20: mol_type = other DNA; note = Primer Khdc4-206-Forward
tacaggcttctccagtcaga

SEQ ID NO: 55 - DNA, 19 bp, synthetic construct
  source 1..19: mol_type = other DNA; note = Primer Khdc4-206-Reverse
tgtccctctctcgctcttt

SEQ ID NO: 56 - DNA, 20 bp, synthetic construct
  source 1..20: mol_type = other DNA; note = Primer Ddx5-208-Forward
ccacactgcaacaccttaca

SEQ ID NO: 57 - DNA, 20 bp, synthetic construct
  source 1..20: mol_type = other DNA; note = Primer Ddx5-208-Reverse
tccttcatgcctcctctacc

SEQ ID NO: 58 - DNA, 18 bp, synthetic construct
  source 1..18: mol_type = other DNA; note = Primer CLK3-intron3_exon5-Forward
ttggtcggatggcgtttc

SEQ ID NO: 59 - DNA, 18 bp, synthetic construct
  source 1..18: mol_type = other DNA; note = Primer CLK3-intron3_exon5-Reverse
ttgccaaaggtgccttca

(19) United States
(12) Patent Application Publication
(10) Pub. No.: US 2025/0223630 A1
(43) Pub. Date: Jul. 10, 2025

(54) IDENTIFYING DISEASE-CAUSING HUMAN DDX41 GENETIC VARIANTS

(71) Applicant: Wisconsin Alumni Research Foundation, Madison, WI (US)

(72) Inventors: Emery Bresnick, Middleton, WI (US); Jeong-Ah Kim, Madison, WI (US); Sunduz Keles, Madison, WI (US)

(73) Assignee: Wisconsin Alumni Research Foundation, Madison, WI (US)

(21) Appl. No.: 19/090,951

(22) Filed: Mar. 26, 2025

Related U.S. Application Data
(63) Continuation-in-part of application No. 18/471,461, filed on Sep. 21, 2023.
(60) Provisional application No. 63/409,311, filed on Sep. 23, 2022.

(51) Int. Cl. C12Q 1/6809; C12N 5/071; C12N 15/10; C12N 15/86; C12Q 1/34; C12Q 1/6886; G01N 15/10; G01N 15/14

(52) U.S. Cl. CPC C12Q 1/6809; C12N 5/0672; C12N 15/1096; C12N 15/86; C12Q 1/34; C12Q 1/6886; G01N 15/14; C12N 2740/00043; C12Q 2600/106; C12Q 2600/156; G01N 2015/1006; G01N 2333/914

(57) ABSTRACT

Described herein is an in vitro genetic rescue assay for identifying a functionally defective DDX41 variant which includes identifying a DEAD-Box Helicase 41 (DDX41) variant of uncertain significance (VUS), infecting a first Ddx41^{+/-} cell with a retrovirus expressing the DDX41-VUS, infecting a second Ddx41^{+/-} cell with a retrovirus expressing a wild type control DDX41, growing the first and second infected cells in culture for a period of time and quantitating mRNA expression of a DDX41-regulated transcript in both the first and second infected cells after the period of time, calculating a differential expression of the DDX41-regulated transcript for the first infected cell compared the second infected cell, and identifying the DDX41-VUS as the functionally defective DDX41 variant wherein a change in the differential expression is 1.5-fold or greater. The identified functionally defective DDX41 variants can be used in methods of monitoring a patient for the development/progression of myeloid malignancy.

### CROSS-REFERENCE TO RELATED APPLICATIONS

This application is a continuation in part of U.S. application Ser. No. 18/471,461, filed Sep. 21, 2023, which claims priority to U.S. Provisional Application 63/409,311 filed on Sep. 23, 2022, which are incorporated herein by reference in their entirety.

### STATEMENT REGARDING FEDERALLY SPONSORED RESEARCH & DEVELOPMENT

This invention was made with government support under DK068634 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on Feb. 26, 2025, is named “SEQ_LIST-107668318-P250197US01.xml” and is 64,028 bytes in size. The Sequence Listing does not go beyond the disclosure in the application as filed.

### FIELD OF THE DISCLOSURE

The present disclosure is related to an in vitro genetic rescue assay for identifying functionally defective DDX41 variants, and the use of the identified variants to monitor a patient for the development/progression of a myeloid malignancy.

### BACKGROUND

Myeloid malignancies are malignant clonal hematopoietic stem cell disorders characterized by ineffective hematopoiesis, marrow dysplasia, and/or peripheral blood cytopenia. Myelodysplastic Syndromes (MDS) are disorders characterized by a failure to produce sufficient healthy, mature blood cells, which have a high propensity for transformation into acute myeloid leukemia (AML). Acute myeloid leukemia (AML) originates from the transformation and clonal expansion of undifferentiated hematopoietic progenitors, characterized by altered growth, differentiation, and proliferation capacities, which result in failure of bone marrow hematopoietic functions. Patients with AML frequently experience infections, easy bleeding, anemia, and organ failure for example.

DEAD-box helicase 41 (DDX41) is a highly conserved member of the RNA helicase family of metazoan proteins that exerts post-transcriptional and translational functions, including the regulation of pre-mRNA splicing and ribosomal RNA transcription. DDX41 senses intracellular DNA in the cGAS-STING pathway, maintains genomic stability at transcriptional loci harboring R-loops regulates inflammatory gene expression and controls snoRNA processing in mouse leukemic stem/progenitor cells.

Heterozygous DDX41 germline variants generate a predisposition for the development of MDS and acute myeloid leukemia AML. Patients present with cytopenias, bone marrow hypocellularity, erythroid dysplasia and/or myeloid malignancy. Although DEAD-box helicase 41 (DDX41) is implicated in oncogenic and innate immune mechanisms, there are many unanswered questions about how the ever-increasing spectrum of genetic variants impact DDX41 activity. Assays to discriminate DDX41 variants of undetermined significance from pathogenic variants, which is fundamental for clinical genetic curation, have not been reported, thus limiting the clinical utility of genetic information.

What is needed are methods of identifying DDX41 pathogenic germline variants.

### BRIEF SUMMARY

In an aspect, an in vitro genetic rescue assay for identifying functionally defective DDX41 variants comprises
identifying a DEAD-Box Helicase 41 (DDX41) variant of uncertain significance (VUS),
infecting a first Ddx41^{+/-} cell with a retrovirus expressing the DDX41-VUS,
infecting a second Ddx41^{+/-} cell with a retrovirus expressing a wild type control DDX41,
growing the first and second infected cells in culture for a period of time and quantitating mRNA expression of a DDX41-regulated transcript in both the first and second infected cells after the period of time, wherein the DDX41-related transcript comprises Sdc1, Fam133b, Gas5, Clk3, or a combination thereof,
calculating a differential expression of the DDX41-regulated transcript for the first infected cell compared to the second infected cell, and
identifying the DDX41-VUS as the functionally defective DDX41 variant wherein a change in the differential expression is 1.5-fold or greater,
wherein a Ddx41^{+/-} cell has a DDX41 expression level that is reduced by at least 50% compared to that of a Ddx41^{+/+} cell.

In another aspect, a method of monitoring a patient for the development/progression of a myeloid malignancy comprises
determining the presence or absence of a functionally defective DDX41 variant determined according to the method described above in a sample from the patient,
optionally determining the presence or absence of the functionally defective DDX41 variant in one or more family members of the patient, and
monitoring the patient and optionally the one or more family members for the development of one or more symptoms of myeloid malignancy when the patient and the one or more family members carry the functionally defective DDX41 variant.

In a further aspect, a method of identifying a patient as at risk for the development/progression of a myeloid malignancy comprises identifying a functionally defective DDX41 variant in the germline of the patient, wherein the functionally defective DDX41 variant is Lys331del.

### BRIEF DESCRIPTION OF THE DRAWINGS

FIGS. 1A-E show family histories and structural predictions of myeloid malignancy associated DDX41 variants. FIG. 1A shows K331del and R293H pedigrees. Probands are indicated with an arrow. FIG. 1B shows the domain structure of human DDX41. Germline and somatic mutations are indicated within the corresponding domains. FIG. 1C shows Sanger sequencing data of probands in each family. FIG. 1D provides the predicted structure of mutant human DDX41 by AlphaFold and 3-D images. FIG. 1E shows the confidence of predicted structure from the AlphaFold analysis.

FIGS. 2A-J show the quantitative genetic rescue assay discriminates DDX41 and human myeloid malignancy-associated variant activities. FIG. 2A illustrates the genetic rescue system. Three CRISPR/Cas9 RNP complexes for Ddx41 were electroporated individually into HoxB-immortalized (hi)-progenitors, and clonal lines were isolated. Retrovirus expressing GFP or GFP and DDX41 or a human disease-associated DDX41 variant were infected into hi-Ddx41^{+/+} and hi-Ddx41^{+/-} cells. GFP-positive cells were isolated by flow cytometry and analyzed by qRT-PCR and RNA-seq. Protein was analyzed by semi-quantitative western blotting. FIG. 2B illustrates exons corresponding to representative domains and target loci of crRNAs on murine Ddx41. FIG. 2C shows Sanger sequencing of genomic DNA at the edited region of hi-Ddx41^{+/-} clonal lines. The crRNA used to generate each clone is indicated in parenthesis. FIG. 2D shows DDX41 protein levels in hi-Ddx41^{+/+} and hi-Ddx41^{+/-} cells were analyzed by semi-quantitative Western blotting using two different antibodies (mouse monoclonal anti-DDX41 antibody (2F4), Novus Biologicals, and a rabbit polyclonal anti-DDX41 antibody). Each clone number indicated corresponds to each clone in FIG. 2C. FIG. 2E shows representative Western blots of Flag-tagged DDX41 and variants and endogenous DDX41 using anti-DDX41 antibody in genetic rescue assay. FIG. 2F shows quantitative analysis of DDX41 protein of FIG. 2E (n=3 per group). FIG. 2G shows the subcellular localization of DDX41 and variants by confocal fluorescence microscope using anti-Flag antibody. FIG. 2H shows mRNAs regulated by DDX41 genetic rescue in hi-Ddx41^{+/-} cells. Heatmap with TPM values of DDX41-regulated mRNAs from total RNA-Seq (n=3 per group). FIG. 2I shows qRT-PCR analysis of DDX41-regulated mRNAs (n=6 per group). Statistics were calculated using one-way ANOVA, followed by Tukey's test; *, P≤0.05; **, P≤0.01; ***, P≤0.001; ****, P≤0.0001. FIG. 2J shows monocytic and granulocytic populations of live GFP+ cells with CD11b and CD115 surface markers detected by flow cytometry (n=6 per group). Statistics were calculated using one-way ANOVA, followed by Tukey's test; *, P≤0.05; **, P≤0.01; ***, P≤0.001; ****, P≤0.0001.

FIGS. 3A-D show qRT-PCR analysis of DDX41-regulated primary transcripts of Clk3 (n=4 per group). qPCR primers on adjacent exons and introns were indicated with arrows in FIG. 3A. Transcript levels were measured in myeloid progenitors (hi-Ddx41^{+/-}; FIGS. 3B,D) and pro-erythroblasts (G1E-ER-G1-Ddx41^{+/-}; FIG. 3C). Statistics were calculated using one-way ANOVA, followed by Tukey's or Sidak test;*, P≤0.05;**, P≤0.01; **, P≤0.001; ****, P≤0.0001.

FIGS. 4A-L show DDX41 regulates Clk3 differential transcript usage in myeloid progenitor cells and DDX41^{+/-} human fibroblasts. (4A) Wiggle plots of Clk3 from RNA-seq (hi-Ddx41^{+/-}_Empty vs. hi-Ddx41^{+/-}_DDX41, G173R or R525H) from 2 replicates (R1 and R2). (4B) Genomic structure of Clk3 transcripts (ENSMUSG00000032316 in GRCm39). (4C and 4D) Heatmap with proportional ratio to total transcripts of Clk3 transcriptional isoforms (4C) and corresponding pie charts (4D). (4E) Splicing events regulated by DDX41 in Clk3-207. (4F and 4G) Detection of intron-retained Clk3 transcript in nuclear and cytoplasmic fractions from cells in the rescue assay with DDX41 and variants using qPCR (F) and RT-qPCR (G; n=4). (H) Western blotting of Lamin B and Tubulin to assess purity of nuclear and cytoplasmic cell fractions. (41) CLK3 mRNA (exon4-5), CLK3 intron-retained transcript, CLK3 pre-mRNA, CLK1, and ACTB mRNA were quantified by RT-qPCR in DDX41 germline-mutated human fibroblasts (p.Lys331del) with age- and gender-matched control (n=4 or 8). (4J) DDX41 genotypes of each iPSC line derived from DDX41 germline-mutated (K331del) or age- and sex-matched DDX41 wild type control skin fibroblasts (upper) and exogenous and endogenous DDX41 levels in rescue assay by Western blotting (lower). (4K) CLK3 mRNA (exon4-5), CLK3 intron-retained transcript, CLK3 pre-mRNA, CLK1, and ACTB mRNA were quantified by RT-qPCR in iPSCs (n=6). (4L) Intron-retained Clk transcripts and mRNAs were analyzed in rescue assay with DDX41 and variants (n=4). RT-qPCR results were presented as box-and-whisker plots with bounds from the 25th to 75th percentiles and the median line, and whiskers ranging from minimum to maximum values. Points represent each replicate. Statistics were calculated using one-way ANOVA, followed by Šídák test; *, P≤0.01; **, P≤0.01; ***, P≤0.001; ****, P≤0.0001.

FIGS. 5A and B show DDX41 increases intron-retained Clk3 transcript and CLK3 protein level upon myeloid differentiation. (5A) CLK3 protein levels in CD11b⁺/CD115⁻ or CD11b⁺/CD115⁺ cells (after 72 h differentiation) in hi-Ddx41 rescue system by Western blotting. (5B) Western blot analysis of CLK3 protein in rescue system in undifferentiated/differentiated hi-cells (K) and quantification (L, n=4).

FIGS. 6A-I show DDX41 confers alternative transcript regulation through CLK3-dependent and -independent mechanisms. (6A) Fold changes and adjusted p-values of DDX41-regulated transcripts from differential transcript expression analysis. (6B) Pie charts depicting the proportion of transcripts in the total transcript pool of Ybx1, Ythdf2, Khdc4, and Ddx5 using TPM values. DDX41-regulated transcripts are indicated as the proportion relative to total transcript (%). (6C) RT-qPCR quantification of transcripts in genetic rescue assay with DDX41, G173R, and K331del in undifferentiated and differentiated cells (n=4). (6D) Genomic and domain structure of Clk3 and Ddx41. Arrowheads, crRNAs for each gene. hi-Ddx41^{+/-};hi-Clk3^{-/-} cells were generated as described in the right box. (6E) Representative Western blot of DDX41 and CLK3 in hi-Clk3^{-/-} and hi-Ddx41^{+/-};Clk3^{-/-} cells. (6F) Flow cytometry analysis for granulocytic and monocytic populations defined by CD11b and CD115 in the rescue assay with hi-Ddx41^{+/-} and hi-Clk3^{-/-} cells. (6G, 6H) hi-Ddx14^{+/-}, two Ddx41^{+/-};Clk3^{-/-} clones (clone1; Cl1 and clone2; Cl2), and Clk3^{-/-} cells were infected with GFP or GFP-DDX41-Flag expressing retrovirus. After three days, GFP-positive cells were sorted and cultured with or without G418 and β-estradiol for three days. Representative Western blot of exogenous DDX41 protein expression with Flag antibody (6G). Asterisks indicate non-specific detection. (6H) RT-qPCR quantification of transcripts (n=7 or 8). (6I) Model for DDX41-dependent establishment of transcript ensembles without or with CLK3. Quantitative flow analysis results were presented as the mean±SEM, and quantitative RT-qPCR results were represented by as box-and-whisker plots with bounds from the 25th to the 75th percentiles and the median line, and whiskers ranging from minimum to maximum values. Statistics were calculated using one-way ANOVA, followed by Šídák test; *, P≤0.01; **, P≤0.01; ***, P≤0.001; ****, P≤0.0001.

The above-described and other features will be appreciated and understood by those skilled in the art from the following detailed description, drawings, and appended claims.

### DETAILED DESCRIPTION

Described herein a genetic rescue assay that provided the identification of two rare DDX41 variants of uncertain significance in two unrelated families as myeloid malignancy-associated DDX41 genetic variants. The genetic rescue assay quantitatively discriminates activities of DDX41 and myeloid malignancy-associated DDX41 genetic variants. The analyses described herein revealed that the variants were impaired in their intrinsic RNA-regulatory activities and to induce monocytic differentiation markers. The quantitative assay described herein can be leveraged to elucidate mechanisms and interventions that promote and/or oppose DDX41 function, thereby influencing DDX41-linked pathogenicity.

In an aspect, an in vitro genetic rescue assay for identifying functionally defective DDX41 variants comprises
identifying a DEAD-Box Helicase 41 (DDX41) variant of uncertain significance (VUS),
infecting a first Ddx41^{+/-} cell with a retrovirus expressing the DDX41-VUS,
infecting a second Ddx41^{+/-} cell with a retrovirus expressing a wild type control DDX41,
growing the first and second infected cells in culture for a period of time and quantitating mRNA expression of a DDX41-regulated transcript in both the first and second infected cells after the period of time, wherein the DDX-41 related transcript comprises Sdc1, Fam133b, Gas5, Clk3, or a combination thereof,
calculating a differential expression of the DDX41-regulated transcript for the first infected cell compared the second infected cell, and
identifying the DDX-41-VUS as the functionally defective DDX41 variant wherein a change in the differential expression is 1.5-fold or greater.

In an aspect, a wherein a Ddx41^{+/-} cell has a DDX41 expression level that is reduced by at least 50% compared to that of a Ddx41^{+/+} cell.

As used herein, a VUS is a variant or unknown or uncertain significance, that is, a genetic variant identified through genetic testing for which the association with disease risk/progression is unclear. A VUS can be benign or pathogenic, for example, and may be identified in a large or small number of individuals. In an aspect, the VUS can be identified in a patient presenting with cytopenia, bone marrow hypocellularity, erythroid dysplasia, and/or myeloid malignancy, but not diagnosed with acute myeloid leukemia.

Cytopenia, for example, is a condition in which one or more blood cell types are lower than normal. Patients who exhibit cytopenia are at risk of progressing to myeloid disease.

Bone marrow hypocellularity is decreased production in one or more hematopoietic cell lineages (myeloid or erythroid). Acute leukemias usually present with hypercellular bone marrow.

Erythroid dysplasia is a condition in which the erythroid cells in the bone marrow are abnormal in size, shape and/or number.

Myeloid malignancies are clonal diseases of hematopoietic stem or progenitor cells. Myeloid malignancies can be present in the bone marrow and in the peripheral blood.

“Acute myeloid leukemia” or “AML”, also known as “acute myelogenous leukemia”, has its general meaning in the art and refers to a cancer of the myeloid line of blood cells, characterized by the rapid growth of abnormal white blood cells that accumulate in the bone marrow and interfere with the production of normal blood cells. AML may be classified using either the World Health Organization classification or the FAB classification.

In an aspect, the DDX41 VUS is identified in a database such as ClinVar or gnomAD.

As described in the Examples, the inventors engineered Hoxb8-immortalized mouse fetal liver progenitor cells, which exhibit a normal myeloid progenitor cell phenotype with CRISPR-Cas9 to generate Ddx41^{+/-} clonal lines with reduced endogenous DDX41 protein levels. WT or mutant DDX41 proteins can be expressed in the Ddx41^{+/-} clonal lines at near-physiological levels and functional consequences e.g., impact on transcript levels. In an aspect, a Ddx41^{+/-} cell has a DDX41 expression level that is reduced by at least 50% compared to that of a Ddx41^{+/+} cell.

In the assay, a first Ddx41^{+/-} cell is infected with a retrovirus expressing the DDX41-VUS, and a second Ddx41^{+/-} cell is infected with a retrovirus expressing a wild type control DDX41. Infection can include standard techniques for infection of cells with a retrovirus. The cells are then grown in culture for a short period of time, such as 2 to 3 days, and then mRNA expression of a DDX41-regulated transcript in both the first and second infected cells after the period of time is quantitated. Based on the RNA-Seq experiments described herein, the DDX41-related transcript comprises Sdc1, Fam133b, Gas5, Clk3, or a combination thereof. A differential expression of the DDX41-regulated transcript for the first infected cell (transcript level in the presence versus absence of DDX41-VUS) is compared to that of the second infected cell (transcript level in the presence versus absence of wild type DDX41) and subjected to statistical analysis. The DDX41-VUS is identified as a functionally defective DDX41 variant wherein a change in the differential expression is 1.5-fold or less than, more specifically 1.8-fold, 2-fold or 2.5-fold less than the differential expression of achieved by expression of wild type DDX41.

In an aspect, the method further includes infecting a third Ddx41^{+/-} cell with a retrovirus expressing a functionally defective variant (e.g., G173R) control DDX41, growing the third infected cells in culture for a short period of time and quantitating mRNA expression of DDX41-regulated genes in the third infected cells, wherein the DDX41-regulated genes comprises Sdc1, Fam133b, Gas5, Clk3, or a combination thereof, and comparing the differential expression of the DDX41-regulated genes for the first infected cell to that of the third infected cell to establish values expected for a functionally defective control. The functionally defective control can be used to supplement the wild type control.

In an aspect, quantitating mRNA expression of a DDX41-regulated transcript comprises reverse-transcriptase PCR. Alternative techniques for quantitating mRNA expression include ribonuclease protection assays and RNA-sequencing.

In order to further verify the functionally defective DDX41 variants flow cytometry may be used. In an aspect, the method further comprises performing flow cytometry on the first infected cells and second infected cells and quantitating a monocytic marker of differentiation of hematopoietic progenitor cells and a granulocytic marker of differentiation of hematopoietic progenitor cells with marker-specific antibodies, wherein a 50% or greater increase in the quantitative level of monocytic marker and 30% or greater decrease in the quantitative level of the granulocytic marker in the first infected cells compared to the second infected cells identifies the functionally defective DDX41 variant as a pathogenic DDX41 variant.

In an aspect, the monocytic markers are CD11b⁺CD115⁺ and/or Ly6C⁺, and the granulocytic markers are CD11b⁺CD115⁻ and/or Ly6G⁺.

Advantageously, the functionally defective DDX41 variants identified by the methods described herein can be used to monitor patients for the development/progression of myeloid malignancy. An exemplary method includes determining the presence or absence of a functionally defective DDX41 variant determined according to the method described herein, optionally determining the presence or absence of the functionally defective DDX41 variant in one or more family members of the patient, and monitoring the patient and optionally the one or more family members for the development of one or more symptoms of myeloid malignancy when the patient and the one or more family members carry the functionally defective DDX41 variant. Exemplary myeloid malignancies include myelodysplastic syndromes (MDS) and acute myeloid leukemia (AML).

Exemplary individuals include those who have a risk factor for developing AML such as being older than 65, being a smoker, having an inherited genetic disorder such as Down syndrome and ataxia telangiectasia, having previously received radiation or chemotherapy, exposure to certain chemicals and having other blood/bone marrow disorders such as aplastic anemia and myelodysplastic syndromes. In addition, having a family member carrying a functionally defective DDX41 variant or other defective variant associated with AML or a family history of myeloid malignancy is also a risk factor.

Exemplary patient samples include blood, bone marrow, fibroblasts, or fractional bone marrow hematopoietic stem/progenitor cells.

Exemplary symptoms of myeloid malignancy include fever, fatigue, irregular heartbeat, dizziness, bone pain, frequent nosebleeds, bleeding and swollen gums, bruising on skin, loss of appetite, excessive sweating, shortness of breath, unexplained weight loss, headaches, diarrhea, menorrhagia, slurred speech, confusion, abdominal swelling, pale skin, seizures, vomiting, loss of balance, facial numbness, blurred vision, or a combination thereof.

In an aspect, wherein when the patient carries the functionally defective DDX41 variant, the method further comprises administering a treatment for the myeloid malignancy.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “chemotherapeutic agent” refers to any chemical agent with therapeutic usefulness in the treatment of cancer. Chemotherapeutic agents as used herein encompass both chemical and biological agents. These agents function to inhibit a cellular activity upon which the leukemic cell depends for continued survival. Categories of chemotherapeutic agents include alkylating/alkaloid agents, antimetabolites, hormones or hormone analogues, and miscellaneous antineoplastic drugs. Most if not all of these drugs are directly toxic to leukemic cells and do not require immune stimulation.

In some embodiments the chemotherapeutic agent is cytarabine (cytosine arabinoside, Ara-C, Cytosar-U®), quizartinib (AC220), sorafenib (BAY 43-9006), lestaurtinib (CEP-701), midostaurin (PKC412), carboplatin, carmustine, chlorambucil, dacarbazine, ifosfamide, lomustine, mechlorethamine, procarbazine, pentostatin, (2′deoxycoformycin), etoposide, teniposide, topotecan, vinblastine, vincristine, paclitaxel, dexamethasone, methylprednisolone, prednisone, all-trans retinoic acid, arsenic trioxide, interferon-alpha, rituximab (Rituxan®), gemtuzumab ozogamicin, imatinib mesylate, melphalan, busulfan (Myleran®), thiotepa, bleomycin, platinum (cisplatin), cyclophosphamide, (Cytoxan®), daunorubicin, doxorubicin, idarubicin, mitoxantrone, 5-azacytidine, cladribine, fludarabine, hydroxyurea, 6-mercaptopurine, methotrexate, 6-thioguanine, or any combination thereof.

In some embodiments, the chemotherapeutic agent is a Bcl-2 inhibitor. In some embodiments, the Bcl-2 inhibitor comprises 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfony-1)-2-(1H-pyrrolo[2,3-b]pyridin-5-yloxy)benzamide (also known as, and optionally referred to herein as, venetoclax, or ABT-199, or GDC-0199) or a pharmaceutically acceptable salt thereof.

In some embodiments, the chemotherapeutic agent is a FLT3 inhibitor. Examples of FLT3 inhibitors include N-(2-diethylaminoethyl)-5-[(Z)-(5-fluoro-2-oxo-1H-indol-3-ylidene)methyl]-2,4-dimethyl-1H-pyrrole-3-carboxamide, sunitinib, also known as SU11248, and marketed as SUTENT® (sunitinib malate); 4-[4-[[4-chloro-3-(trifluoromethyl)phenyl]carbamoylamino]phenoxy]-N-methy-1-pyridine-2-carboxamide, sorafenib, also known as BAY 43-9006, marketed as NEXAVAR® (sorafenib); (9S,10R,11R,13R)-2,3,10,11,12, 13-hexahydro-10-methoxy-9-methyl-11-(methyl-amino)-9,13-epoxy-1H,9H-diindolo [1,2,3-gh:3′,2′,1′-lm]pyrrolo[3,4-j][1,7]benzodiamzonine-1-one, also known as midostaurin or PKC412; (5 S,6 S,8R)-6-hydroxy-6-(hydroxymethyl)-5-methyl-7,8,14,15-tetrahydro-5H-16-oxa-4b,8a,14-triaza-5,8-methanodibenzo[b,h]cycloocta[jkl]cyclopenta[e]-as-indacen-13 (6H)-one, also known as lestaurtinib or CEP-701; 1-(5-(tert-Butyl)isoxazol-3-yl)-3-(4-(7-(2-morpholinoethoxy)benzo[d]imida-zo[2,1-b]thiazol-2-yl)phenyl)urea, also known as Quizartinib or AC220; 1-(2-{5-[(3-methyloxetan-3-yl)methoxy]-1H-benzimidazol-1-yl}quinolin-8-yl-)piperidin-4-amine, also known as Crenolanib or CP-868,596-26

In some embodiments, the chemotherapeutic agent is an IDH (isocitrate dehydrogenase) inhibitor. In some embodiments, the IDH inhibitor is a member of the oxazolidinone (3-pyrimidinyl-4-yl-oxazolidin-2-one) family, and is a specific inhibitor of the neomorphic activity of IDH1 mutants and has the chemical name (S)-4-isopropyl-3-(2-(((S)-1-(4 phenoxyphenyl)ethyl)amino)pyrimidin-4-yl)oxazolidin-2-one.

Another treatment for AML is a stem cell transplant. In certain aspects, stem cell transplants allow for higher doses of chemotherapeutic agents to be administered. Exemplary stem cell transplants include allogenic stem cell transplants (typically from a tissue matched donor) and autologous stem cell transplants (a patient's own stem cells).

In a specific aspect, a method of identifying a patient as at risk for the development/progression of a myeloid malignancy comprises identifying a functionally DDX41 variant in the germline of the patient, wherein the functionally defective DDX41 variant is Lys331del.

In an aspect, the method further comprises administering a treatment for the myeloid malignancy such as chemotherapy or a stem cell transplant.

The invention is further illustrated by the following non-limiting examples.

### EXAMPLES

### Methods

Patients and data collection: Families 1 and 2 were identified in University of Wisconsin/UW Health Hereditary Hematology and Bone Marrow Failure Clinic. Informed consent was obtained from probands and available close relatives or their healthcare proxy for deceased individuals. DDX41 germline variants were identified through CLIA-certified clinical genetic testing and confirmed and segregated in available family members via Sanger sequencing. Medical and family history were obtained by patient and family member interviews and medical record review. This study was approved by University of Wisconsin-Madison Health Sciences Institutional Review Board and conducted in accordance with the Declaration of Helsinki.

Protein structure prediction: AlphaFold was used to predict DDX41 structure. The DDX41 model was obtained from AlphaFold database (AF-Q9UJV9). DDX41 mutant structural models were generated with AlphaFold v2.1.0 Colab.

HoxB8-immortalized (hi)-Ddx41 heterozygous mutant (Ddx41^{+/-}) cells: To generate HoxB8-immortalized (hi)-Ddx41 mutant progenitors, CRISPR/Cas9 RNP complexes were introduced individually by electrophoresis into hi-WT progenitors 318 described in the art. ER-HoxB8-immortalized (hi)-hematopoietic progenitors were generated by retroviral infection of estrogen-regulated HoxB8 into primary Lin-cells isolated from E14.5 mouse fetal liver. The sex of the cells was male, identified by PCR analysis of the DDX3y gene with genomic DNA. For targeting Ddx41, three crRNAs (A;5′-AGATGAGGACGACATCCCGC-3′ (SEQ ID NO: 1), B;5′-TGATCGGCATTGCCTTCACG-3′ (SEQ ID NO: 2), and C;3′-ATGCTCAGGACATAACGCGG-5′ (SEQ ID NO: 3), Integrated DNA Technologies; Coralville, IA, USA) targeting distinct exons were annealed to tracrRNA to generated guide RNAs which were then assembled into RNP complexes with sNLS-spCas9-sNLS (Aldevron (Madison, WI, USA). Each RNP complex was introduced into 2×105 hi-progenitor cells using 4D Nucleofector™ with P3 Primary Cell 4D-Nucleofector® X Kit (Lonza; Basel, Switzerland). 72 hours post-electroporation, clones were isolated by limiting dilution (0.5 cell/well of a 96-well plate). Clones were cultured until colonies became visible and where then transferred to larger wells. Sanger sequencing of genomic DNA was used to establish genotype, and sequencing data was analyzed by SnapGene Viewer (San Diego, CA, USA). Cells were cultured in OPTI-MEM™ supplemented with 10% FBS, 1% penicillin streptomycin, 1% SCF-conditioned medium, 28.6 μM β-mercaptoethanol, 1 μM estradiol, and 400 μg/ml G418 in a humidified 5% CO₂ incubator at 37° C.

DDX41 rescue assay: DDX41 and human myeloid malignancy-associated DDX41 variants were expressed from the MSCV-PIG retroviral expression vector. Retroviruses were packaged in 293T cells and retrovirus-containing supernatants were collected 48 h post-transfection. hi-Ddx41^{+/+} and hi-Ddx41^{+/-} hematopoietic progenitors were infected with retrovirus expressing GFP alone or GFP and DDX41 (WT or variants). Cells were transferred to IMDM containing 2% FBS and incubated with infectious supernatant by spinoculation for 90 min at 2,800 rpm at 30° C. Cells were cultured for 2 days in immortalized cell culture media described above. GFP+ cells were isolated by fluorescence-activated cell sorting with FACSAria™ cell sorter (BD Biosciences; Franklin Lake, NJ, USA). RNA and protein of sorted GFP-positive cells was analyzed by RNA-seq, RT-qPCR and semi-quantitative Western blotting.

Semi-quantitative Western blotting: Cells were washed with ice-cold PBS and boiled for 10 min in 2× sodium dodecyl sulfate (SDS) lysis buffer (50 mM Tris (pH 6.8), 2% β-mercaptoethanol, 2% SDS, 0.1% bromophenol blue, 10% glycerol). Proteins were analyzed by western blotting with ECL 2 (Thermo Fisher Scientific; Waltham, MA, USA) and West Femto (Thermo Fisher Scientific) with rabbit monoclonal anti-Flag (Cell Signaling Technology; Danvers, MA, USA), mouse monoclonal anti-DDX41 (Novus Bioscience; Littleton, CO, USA), rabbit polyclonal anti-DDX41 antibody generated against purified full-length DDX41 protein (Antigen designed by inventors, produced by Cocalico Biologicals, Inc.; Stevens, PA, USA), mouse monoclonal anti-beta-actin (Cell Signaling Technology) or anti-GAPDH (Cell Signaling Technology). Blots were developed using LI-COR Odyssey® Imaging System (LI-COR Biosciences; Lincoln, NE, USA) and quantified by Image Studio Lite (version 5.2.5) (LI-COR Biosciences). Quantification is represented as mean with SD. Statistical analyses were conducted using ANOVA tests (significance cutoff of p value <0.05) as calculated using Prism software (GraphPad Software; San Diego, CA, USA).

Immunofluorescence: hi-Ddx41^{+/+} cells were infected with empty retrovirus or retroviruses expressing DDX41 or variants, collected on poly-L-lysine coated slides (Electron Microscopy Sciences; Hatfield, PA, USA) and fixed with 3.7% paraformaldehyde in PBS for 10 min at room temperature. Slides were washed with PBS and permeabilized with 0.2% Triton™ X-100 for 10 min at room temperature. Washed slides were blocked with 3% BSA in PBS containing 0.1% Tween™ 20 for 1 h at room temperature and incubated with rabbit anti-Flag (Cell Signaling Technology) in 3% BSA at 4° C. overnight. After washing, slides were incubated with Alexa Fluor® 594 secondary antibody (Invitrogen; Waltham, MA, USA) for 1 h at room temperature, washed and mounted using Vectashield® mounting medium with DAPI (Vector Laboratories; Burlingame, CA, USA). Images were acquired with a Nikon A1R-S confocal microscope (Nikon; Minato City, Tokyo, Japan).

RNA-seq with DDX41 rescue assay: Three biological replicates of hi-Ddx41^{+/+} cells infected with empty vector and hi-Ddx41^{+/-} cells infected with empty vector or retroviruses expressing DDX41 or G173R or R525H were harvested and sorted for GFP+ cells on a FACSAria™ II instrument (BD Biosciences). RNA was purified using an RNAeasy® Micro Kit (Qiagen; Hilden, Germany). Library sequencing through Illumina® TruSeq™ Stranded Total RNA (rRNA reduction) was conducted by the University of Wisconsin-Madison Gene Expression Center and sequenced using an Illumina® NovaSeq™ 6000. Reads were aligned by STAR (version 2.5.2b) to the mouse genome (version mm39) with GENCODE basic gene annotations (version M22). Gene expression levels were quantified by RSEM (version 1.3.0), and differential expression was analyzed by edgeR (version 3.30.3).

qRT-PCR: Total RNA was purified from 2-5×105 cells with TRIzol™ (Invitrogen) and 1-2 μg RNA was treated with DNase I (Thermo Fisher Scientific) for 15 min at room temperature. After heat inactivation of DNase I with EDTA for 10 min at 65° C., 0.5-1 μg RNA was incubated with a 4:1 mixture of oligo(dT) primers and random hexamer at 68° C. for 10 min. RNA/primers were incubated with Moloney murine leukemia virus reverse transcription (M-MLV RT) (Thermo Fisher Scientific), 5× first strand buffer (Thermo Fisher Scientific), 10 mM dithiothreitol (Thermo Fisher Scientific), RNAsin® (Promega), and 0.5 mM deoxynucleoside triphosphates (New England Biolabs; Ipswich, MA, USA) at 42° C. for 1 h and then heat inactivated at 95° C. for 5 min. Quantitative gene expression analyses were conducted by real-time RT-PCR using Power SYBRTM Green Master Mix (Applied Biosystems; Waltham, MA, USA) and analyzed on a ViiA™ 7 Real-Time PCR System (Applied Biosystems). Control reactions without M-MLV RT yielded little to no signal. Relative expression of mRNA was determined from a standard curve of serial dilutions of cDNA samples and values were normalized to Hprt RNA expression. Quantitative RT PCR results were presented as box and whisker plots. Statistical comparisons were conducted using ANOVA test (significance cutoff of P value <0.05) as calculated using Prism software (GraphPad Software).

Cell differentiation and flow cytometry: hi-Ddx41^{+/+} and hi-Ddx41^{+/-} cells were infected with retroviruses expressing DDX41 or variants. One day post-infection, cells were washed with ice-cold PBS and resuspended in differentiation medium (OPTI-MEM™ supplemented with 10% FBS, 1% penicillin streptomycin, 1% SCF-conditioned medium, 1% IL-3-conditioned medium, and 28.6 μM beta-mercaptoethanol). Cells were cultured for 3 days at 37° C. For analysis of monocytic and granulocytic populations by flow cytometry, cells were washed with ice-cold PBS containing 2% FBS and 2 mM EDTA, and live/dead cell staining was conducted using Ghost Dye™ Red 780 (Tonbo Biosciences, San Diego, CA, USA) at 4° C. for 15 min, followed by washing with PBS containing 2% FBS and 2 mM EDTA. Surface antigens were stained using 1:200 diluted combinations of APC-CD11b (BioLegend) and PE-CD115 (BioLegend) in PBS with 2% FBS and 2 mM EDTA at 4° C. for 30 min. After staining, cells were washed with ice-cold PBS containing 2% FBS and EDTA and analyzed on an Attune™ NxT Flow Cytometer (Thermo Fisher Scientific). Differentiated cell populations were analyzed using FlowJo v10.8.0 software (BD Biosciences).

TABLE 1
PRIMERS

| Target | Sequence | SEQ ID NO: |
|---|---|---|
| DDX41 Exon9-Forward | ATCATCTGCCCCTCGGTAAG | 4 |
| DDX41 Exon9-Reverse | CCCCAACTAACCTCCCCATT | 5 |
| DDX41 Exon10-Forward | ATACATAGGGCAGGTGGTGG | 6 |
| DDX41 Exon10-Reverse | AGCTGAGCAACTGAGACACA | 7 |
| Sdc1-exon3_4-Forward | GAGGATGGAACTGCCAATCA | 8 |
| Sdc1-exon3_4-Reverse | CCCAGATGTTTCAAAGGTGAAG | 9 |
| Sdc1-exon4_5-Forward | GGTGCTTCTCAGAGCCTTT | 10 |
| Sdc1-exon4_5-Reverse | AGGCACACAGCAAAGATGA | 11 |
| Clk3-exon4_5-Forward | GGCGATTGGCTCCAAGA | 12 |
| Clk3-exon4_5-Reverse | AGCACTCCACCACCTTG | 13 |
| Clk3-exon9_10-Forward | CCATGAACATCACACCACCA | 14 |
| Clk3-exon9_10-Reverse | CTCAAAGAGAATGCAGCCGATA | 15 |
| Fam133b-exon6_7-Forward | TCTGATTCTTCCAGCAGTTCTT | 16 |
| Fam133b-exon6_7-Reverse | AGGAGACTTATGGCAACGG | 17 |
| Fam133b-exon7_8-Forward | CGTTGCCATAAGTCTCCTGA | 18 |
| Fam133b-exon7_8-Reverse | TTTCTCTTTCAGTTACATCCTTGG | 19 |
| Gas5-exon4_6-Forward | GTCAGGAAGCTGGATAACAGAG | 20 |
| Gas5-exon4_6-Reverse | AGCCTCAAACTCCACCATTT | 21 |
| Gas5-exon6_8-Forward | GGTGGAGTTTGAGGCTGGATA | 22 |
| Gas5-exon6_8-Reverse | CCAAGCAAGCCAGCCAA | 23 |

### Star Methods

### Cell Lines

HoxB8 immortalized (hi)-DDX41 heterozygous mutant (Ddx41^{+/-}), Clk3 nullizygous (Clk3^{-/-}), and Ddx41 and Clk3 double-mutant cell (Ddx41^{+/-};Clk3^{-/-}): To generate HoxB8-immortalized Ddx41 and Clk3 double-mutant progenitor cells, CRISPR/Cas9 RNP complex was introduced by electrophoresis into hi-WT progenitor cells as described in the art. ER-HoxB8 immortalized (hi)-hematopoietic progenitor cells were generated by retroviral infection of b-estradiol-regulated HoxB8 into primary Lin³¹ cells isolated from WT mouse fetal liver. The sex of the cells was male, identified by genomic DNA PCR of the Ddx3y gene. Three crRNAs were from IDT™ and sNLS-spCas9-sNLS was from Aldevron. Each RNP complex contained Clk3 crRNAs and tracrRNA introduced into 2×105 hi-progenitor cells using 4D-Nucleofector™ with P3 Primary Cell 4D-Nucleofector® X Kit. 72 h post-electroporation, clones were isolated by limiting dilution (0.5 cell/well of a 96-well plate). Clones were cultured until colonies appeared and transferred to larger wells. Genomic DNA was subjected to Sanger sequencing, and the results were analyzed with SnapGene Viewer. Double-mutant clones were obtained by introducing Ddx41 crRNAs into hi-Clk3^{-/-} clones identical to that described above. Cells were cultured in OPTI-MEM supplemented with 10% FBS, 1% penicillin-streptomycin, 1% SCF-conditioned medium, 28.6 μM β-mercaptoethanol, 1 μM β-estradiol, and 400 μg/ml G418 (immortalized cell culture media). Cells were cultured in a humidified 5% CO₂ incubator at 37° C.

Primary human skin fibroblasts: Primary human skin fibroblasts were cultured from 3 mm punch skin biopsies from: 1) a male identified in The University of Wisconsin/UW Health Hereditary Hematology and Bone Marrow Failure Clinic to carry a heterozygous germline pathogenic variant in DDX41 (K331del) and 2) an age-matched male sequenced for pathogenic variants in 120 inherited bone marrow failure and cancer predisposition genes (including DDX41) and found to be wild-type for all sequenced genes. Informed consent for use of skin fibroblasts and iPSC generation for research purposes was obtained from both subjects. DDX41 germline variant status was determined through CLIA-certified clinical genetic testing and confirmed in skin fibroblast samples used in this study via Sanger sequencing. This study was approved by University of Wisconsin-Madison Health Sciences Institutional Review Board and conducted in accordance with the Declaration of Helsinki. Fibroblasts were initially cultured and expanded in AmnioMax™ Medium+C-100 Supplement (Gibco/ThermoFisher). Control and DDX41 K331del fibroblasts at passage two to four were utilized for subsequent experiments or iPSC generation. For RNA-based experiments, 2×10⁶ fibroblast cells were collected from four separate, expanded culture flasks at similar passage number from the DDX41 K331del and wild type control sample. RNA was isolated and analyzed by RT-qPCR.

Human induced pluripotent stem cells (iPSCs): Human iPSCs from DDX41 K331del germline-mutated skin fibroblasts were reprogrammed using the CytoTune™-iPS 2.0 Sendai Reprogramming Kit (ThermoFisher Scientific). For reprogramming, 1×10E⁵ fibroblasts (WT or DDX41 mutant) were plated in AmnioMax™ C-100 medium+C-100 supplement (Gibco/ThermoFisher) and allowed to adhere overnight. For transduction, the medium was replaced with 1 ml of fibroblast medium containing 10 ml of the Sendai viruses expressing KOS (KLF4, OCT3/4-SOX2) MYC and KLF4 and incubated overnight. Fresh medium was added the following day, and cells were cultured for another 5 days in AmnioMax™ medium. Cells were transferred to mouse embryonic feeder layers (MEFs; WiCell) and cultured in mTESR™ Plus medium (STEMCELL) until colonies appeared. Individual colonies were picked and further cultured on Cultrex Reduced Growth Factor Basement Membrane Extract (Cultrex; R&D systems)-coated plates. Expression of pluripotency markers for SOX2, NANOG and OCT3/4 was analyzed by immunofluorescence using anti-human OCT3/4-clone C10 (SantaCruz), anti-human Nanog-clone D73G4 (Cell Signaling), anti-human SOX2-clone D6D9 (Cell Signaling). Selected wild-type and DDX41 mutant iPSC clones were cultured with mTeSR™ plus in Cultrex-coated cell culture plates in feeder-free conditions for further examination.

### Method Details

Genetic rescue assay of hi-progenitor cells: DDX41 or mutants were expressed in hi-Ddx41^{+/+}, hi-Ddx41^{+/-}, hi-Clk3^{-/-}, or hi-Ddx41^{+/-}; Clk3^{-/-} hematopoietic progenitors by infection of retrovirus expressing DDX41 and pathogenic variants, G173R and R525H. Retrovirus was packaged in 293T cells with MSCV-PIG containing DDX41, G173R, R525H, or K331del and pCL-Eco plasmids, and supernatants containing each virus were collected 48 h post-transfection. Cells were transferred to IMDM containing 2% FBS and incubated with infectious supernatant by spinoculation for 90 min at 2800 rpm at 30° C. Cells were cultured for 3 days in immortalized cell culture media described above. GFP⁺ cells were sorted by fluorescence-activated cell sorting with FACSAria™ cell sorter (BD Biosciences). Sorted cells were washed twice with PBS and differentiated for 3 days by the removal of β-estradiol. RNA and protein of sorted GFP⁺ cells were analyzed by RNA-seq, RT-qPCR and Western blotting.

Subcellular fractionation: Cells were isolated by centrifugation at 1200 rpm for 6-8 min at 4° C. and washed with 0.5 mL PBS in 1.5 ml tubes. Cells were re-isolated by centrifugation at 1500 rpm for 5 min at 4° C. Cells were lysed with 1.5-fold pellet volumes of cell lysis buffer (50 mM Tris-HCl, pH 8.0, 10 mM EDTA, pH 8.0, 1% SDS). After incubation on ice for 10 min, cells were subjected to centrifugation at 1200 rpm for 5 min at 4° C. The supernatant (cytoplasmic fraction) was taken with a pipette tip immediately, being careful not to disturb the pellet (nuclear fraction).

Genetic rescue assay with Human induced-pluripotent stem cells (iPSCs): Human iPSCs were generated from DDX41 K331del germline mutated fibroblasts by Dr. Igor Slukvin's laboratory. Selected wild-type and DDX41 mutant iPSC clones were cultured with mTeSR™ plus in Cultrex-coated cell culture plates in feeder-free conditions for further examination. Lentivirus was packaged in 293T with DDX41 cDNA containing pCDH-CMV-EF1-GFP, pMD2G, and psPax2 plasmids, and supernatants were collected 48 h after transfection. GFP alone or DDX41 expressing lentivirus was transduced into human iPSCs with 10 μg/ml polybrene in mTeSR™ plus. Supernatants were changed after a day with fresh mTeSR™ plus. Cells were cultured for 2 days more in mTeSR™ plus. RNA and protein from sorted GFP⁺ cells were analyzed by RT-qPCR and Western blotting.

Western blotting: Protein was extracted from cells and analyzed by Western blotting. Samples were washed with ice-cold PBS and boiled for 10 min in sodium dodecyl sulfate (SDS) lysis buffer (50 mM Tris (pH 6.8), 2% β-mercaptoethanol, 2% SDS, 0.1% bromophenol blue, 10% glycerol). Proteins were analyzed by semi-quantitative Western blotting with ECL2 (Thermo Fisher Scientific) and West Femto (Thermo Fisher Scientific) with rabbit monoclonal anti-Flag (Cell Signaling Technology), rabbit monoclonal anti-HA (Cell Signaling Technology), mouse monoclonal anti-DDX41 (Novus Bioscience), rabbit polyclonal anti-DDX41 antibody (custom-made with full-length DDX41 protein by Cocalico Biologicals, Inc.), mouse monoclonal anti-βactin (Cell Signaling Technology), rabbit monoclonal anti-LaminB (abcam) or anti-Tubulin (Cell Signaling Technology). Blots were developed by LI-COR Odyssey Imaging System (LI-COR Biosciences) and quantified by Image Studio Lite (version 5.2.5) (LI-COR Biosciences). Quantification was represented as mean with SEM and statistical analyses were performed using ANOVA tests (significance cutoff of P value <0.05) as calculated using Prism software (GraphPad Software).

qRT-PCR: Total RNA was purified from 2-5×10⁵ cells with TRIzol (Invitrogen) and 1-2 μg RNA was treated with DNase I (Thermo Fisher Scientific) for 15 min at room temperature. After heat inactivation of DNase I with EDTA for 10 min at 65° C., 0.5-1 μg RNA was incubated with a 4:1 mixture of oligo(dT) primers and random hexamer at 68° C. for 10 min. RNA/primers were incubated with Moloney murine leukemia virus reverse transcription (M-MLV RT) (Thermo Fisher Scientific), 5× first strand buffer (Thermo Fisher Scientific), 10 mM dithiothreitol (Thermo Fisher Scientific), RNAsin (Promega), and 0.5 mM deoxynucleoside triphosphates (New England Biolabs) at 42° C. for 1 h and then heat inactivated at 95° C. for 5 min. Quantitative gene expression analyses were performed by real-time RT-PCR using Power SYBR Green Master Mix (Applied Biosystems) and analyzed on a ViiA 7 Real-Time PCR System (Applied Biosystems). Control reactions without M-MLV RT yielded little to no signal. Relative expression of RNA was determined from a standard curve of serial dilutions of cDNA samples, and values were normalized with Hprt for mouse or HPRT for human RNA expression. Quantitative RT-PCR results were presented by box and whiskers plot. Statistical comparisons were performed using ANOVA test (significance cutoff of P value <0.05) as calculated using Prism software (GraphPad Software).

Cell differentiation and flow cytometry: hi-Ddx41^{+/-}, Clk3^{-/-}, or Ddx41^{+/-}; Clk3^{-/-} cells were infected with retroviruses expressing GFP, GFP-DDX41 or variants. One day post-infection, cells were washed with PBS twice and resuspended in differentiation medium (OPTI-MEM supplemented with 10% FBS, 1% penicillin-streptomycin, 1% SCF-conditioned medium, 1% IL-3-conditioned medium, and 28.6 μM β-mercaptoethanol). Cells were cultured for 3 days at 37° C. For analysis of monocytic and granulocytic populations by flow cytometry, cells were washed with ice-cold PBS containing 2% FBS and 2 mM EDTA, and live/dead cell staining was conducted using Ghost Red Dye 780 (Tonbo Biosciences, San Diego, CA, USA) at 4° C. for 15 min, followed by washing with PBS containing 2% FBS and 2 mM EDTA. Surface antigens were stained using 1:200 diluted combinations of APC-CD11b (BioLegend) and PE-CD115 (BioLegend) in PBS with 2% FBS and 2 mM EDTA at 4° C. for 30 min. After staining, cells were washed with ice-cold PBS containing 2% FBS and EDTA, and analyzed on an Attune™ NxT Flow Cytometer (Thermo Fisher Scientific). Differentiated cell populations were analyzed using FlowJo v10.8.0 software (BD Biosciences).

Differential transcript expression (DTE) and differential transcript usage (DTU) analyses of RNA-seq data: Four biological replicates of hi-Ddx41^{+/-} cells infected with empty vector, DDX41 WT, G173R, or R525H were harvested and sorted for GFP⁺ cells on a FACSAria II instrument (BD Biosciences). RNA was purified using an RNAeasy Micro Kit (Qiagen). Library sequencing through Illumina® TruSeq Stranded Total RNA (rRNA reduction) was prepared by the University of Wisconsin-Madison Gene Expression Center and sequenced using an Illumina® NovaSeq 6000 sequencer. Reads were aligned by STAR (version 2.7.3a) to the mouse genome (version mm39) with GENCODE basic gene annotations (version M22) as published before.

DTE analysis was conducted using the R package DESeq2. False discovery rate was controlled at 0.1 with the Benjamini-Hochberg procedure and a cutoff of 0.5 was applied to absolute log fold changes. DTU analysis was carried out with the R package DTUrtle. The analysis workflow began with generating a transcript-to-gene map using the one_to_one_mapping function, which annotated each transcript with its corresponding gene and transcript identifiers based on the mouse vM32 GTF file. Statistical analysis was performed using DRIMseq in DTUrtle. Differential expressed and used transcripts were visualized by VolcaNoseR. We set the minimum transcript proportion change to 0.1, and FDR control threshold to 0.1. The adjusted p-values were calculated with StageR⁵ based on the instructions provided by DTUrtle. Differential alternative transcriptional regulation (ATR) analysis was carried out by SURF with an FDR control of 0.05.

Protein analysis: Western blot signals, normalized to the hi-Ddx41^{+/-} empty control or DDX41 rescued hi-Ddx41^{+/-} values, were presented as box-and-whisker plots with bounds from the 25th to the 75th percentiles and the median line, and whiskers ranging from minimum to maximum values. Points represent each replicate. Statistical comparisons were performed using one-way ANOVA, followed by Šídák test using Prism software (GraphPad Software); *, P≤0.01; **, P≤0.01; ***, P≤0.001; ****, P≤0.0001.

qRT-PCR analysis: Quantitative RT-qPCR results were presented as box-and-whisker plots with bounds from the 25th to the 75th percentiles and the median line, and whiskers ranging from minimum to maximum values. Points represent each replicate. Statistical comparisons were performed using one-way ANOVA, followed by Šídák test using Prism software (GraphPad Software); *, P≤0.01; **, P≤0.01; ***, P≤0.001; ****, P≤0.0001.

Flow cytometry: Quantitative flow analysis results were presented as the mean±SEM. For multiple comparisons to hi-Ddx41^{+/-} GFP control or DDX41 rescued hi-Ddx41^{+/-}, one-way ANOVA was used, followed by Šídák test using Prism software (GraphPad Software); *, P≤0.01; **, P≤0.01; ***, P≤0.001; ****, P≤0.0001.

### Example 1: Identification of DDX41 VUS

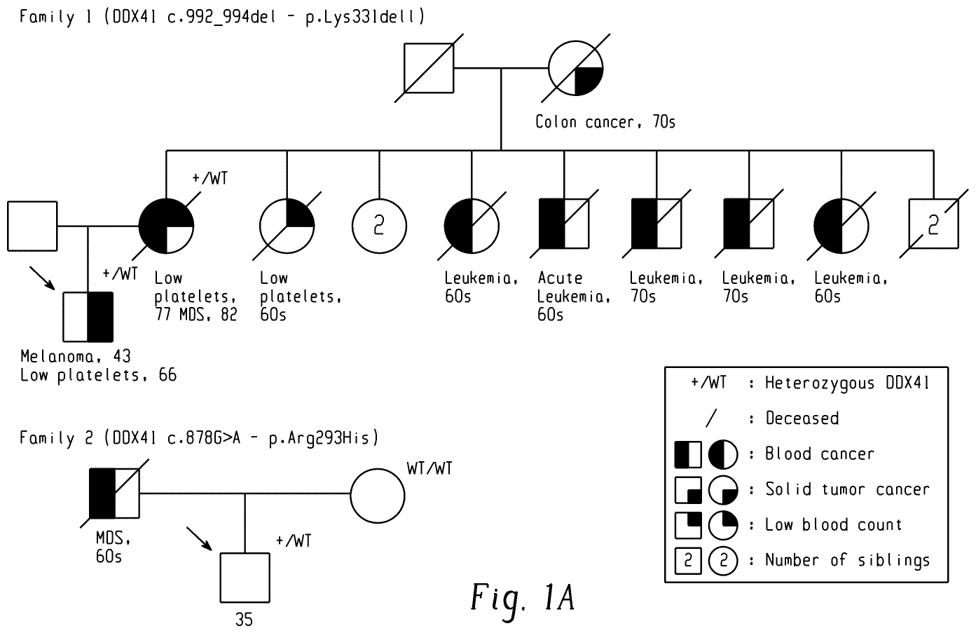

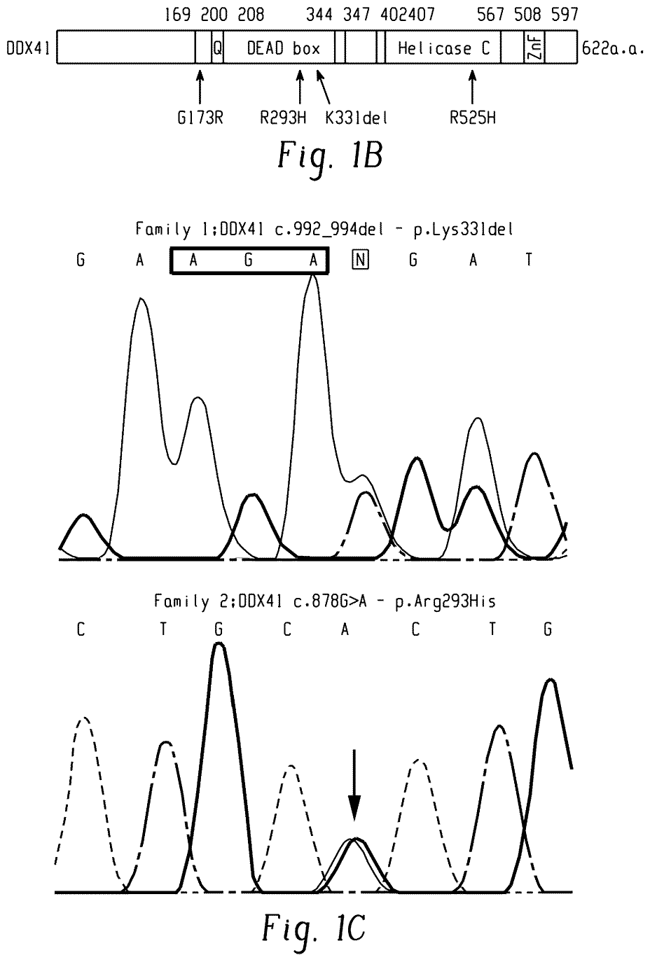

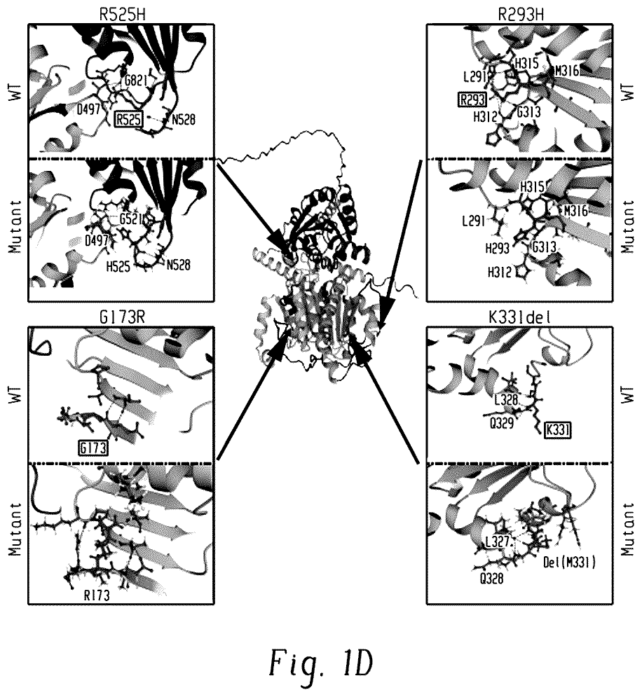

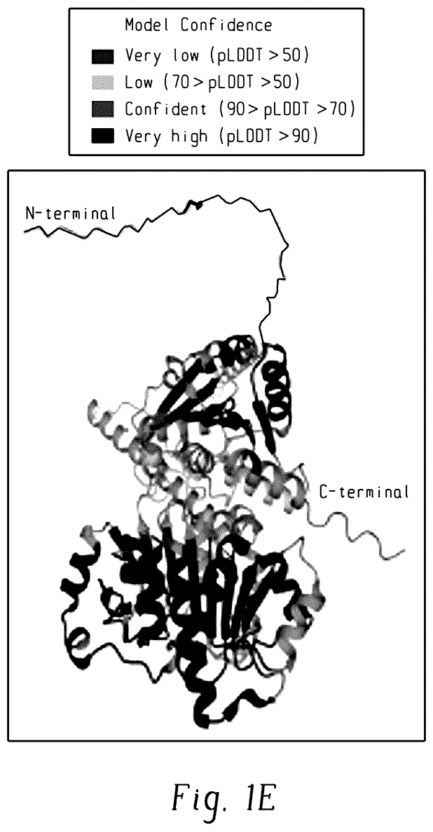

Two rare DDX41 variants of uncertain significance were identified in two unrelated families (FIG. 1A). In family 1 (FIG. 1A upper), the proband with p.Lys331del was diagnosed with melanoma at age 43 and low platelets in the context of a normal bone marrow examination at age 66. His mother was diagnosed with low platelets at age 77 and MDS with excess blasts-1 with a normal karyotype and DDX41 p.Lys331del (VAF 83%) at age 82. Her three brothers and three sisters were diagnosed with low platelets or leukemia after their 60s. In family 2 (FIG. 1A lower), the proband carried germline p.Arg293His. His father was diagnosed with normal karyotype MDS at age 62. He quickly progressed to AML and passed away after allogeneic stem cell transplantation. Both variants occur within the well-conserved DEAD-box domain (FIG. 1B). Sanger sequencing of genomic DNA confirmed DDX41 sequence variation (FIG. 1C). These residues are evolutionarily conserved.

To predict whether deletion or substitution of these residues may disrupt DDX41 three-dimensional structure, AlphaFold was utilized. As with the previously reported DDX41 recurrent germline variant (Gly173Arg) and the common somatic variant (Arg525His), the gross structural features of the variants are predicted to resemble wild type (WT) DDX41 (FIG.1D). The modeling predicts that DDX41 Arg293 forms hydrogen bonds surrounding Leu291, His315, Met316, His312, and Gly313 residues. With the histidine substitution of this residue (R293H), the guanidino group is replaced with an imidazole ring, which is predicted to abrogate hydrogen bonding with Leu291, His312, and Gly313 residues (FIG.1E, upper right). Hydrogen bond disruption was also predicted for the p.Arg525His (R525H) variant. While Arg525 is predicted to form hydrogen bonds with Gly521, Asp497, and Asn528, the Arg525His variant might lose hydrogen bonds with Gly521 and Asp497 (FIG.1E, upper left). Lys331 can form hydrogen bonds with Leu328 and Gln329. The lysine 331 deletion (K331del) is predicted to disrupt hydrogen bonding with Gln329 (FIG.1E, lower right). Since intramolecular hydrogen bonds stabilize protein conformations, in principle, altered intramolecular interactions may alter DDX41 function. Although the structural predictive analysis did not reveal whether the variant was pathogenic, based on the prediction of an altered intramolecular interaction, it seems logical that this could affect DDX41 function.

### Example 2: Production of DDX41^{+/-} Clonal Lines

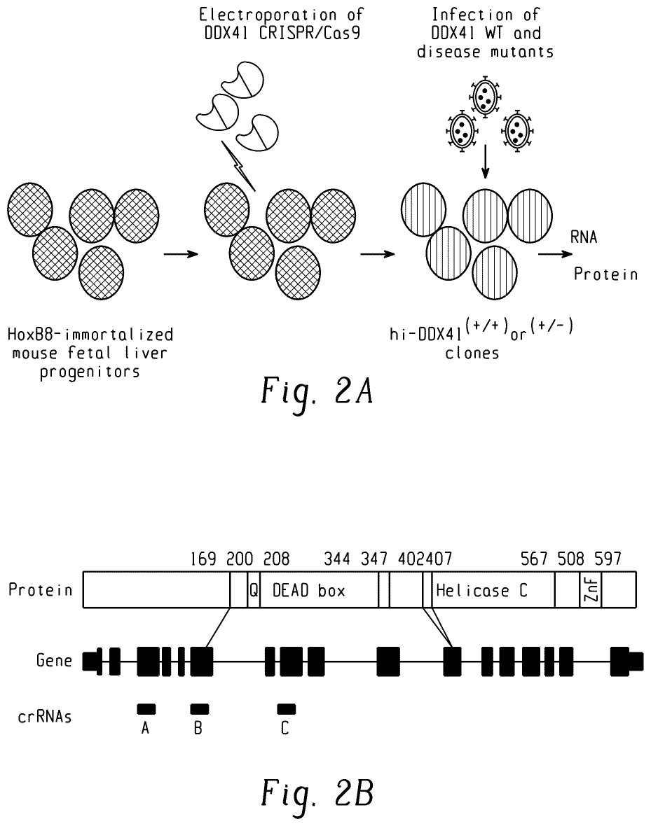

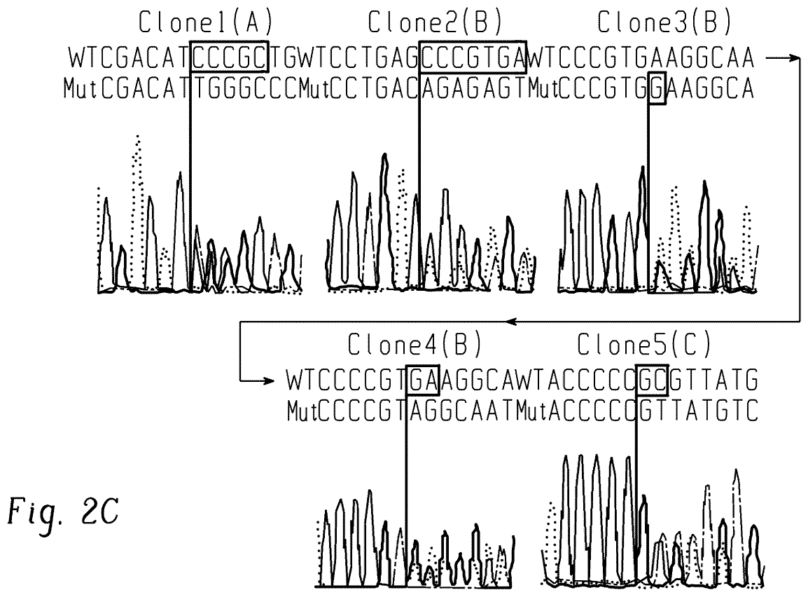

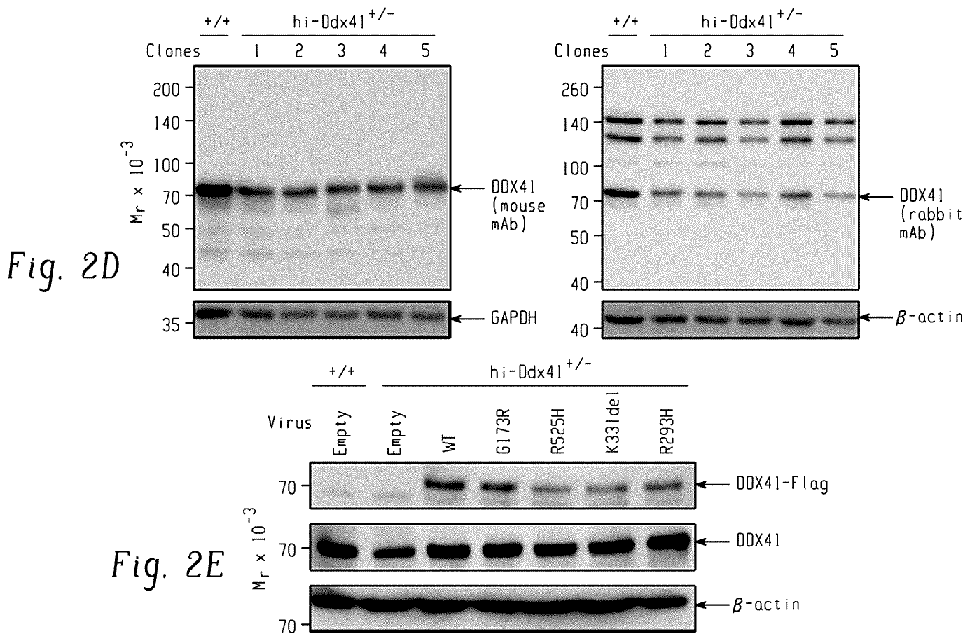

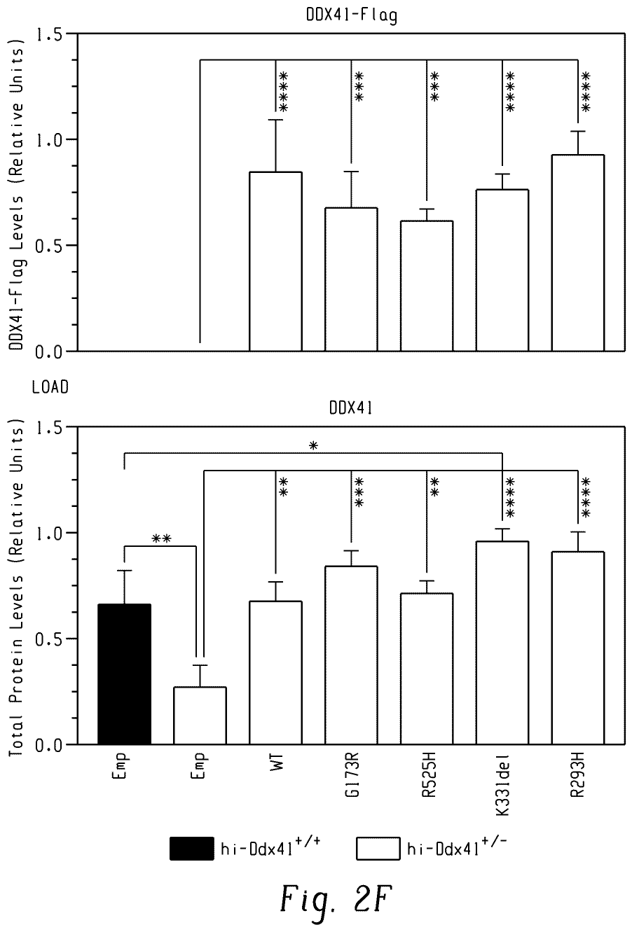

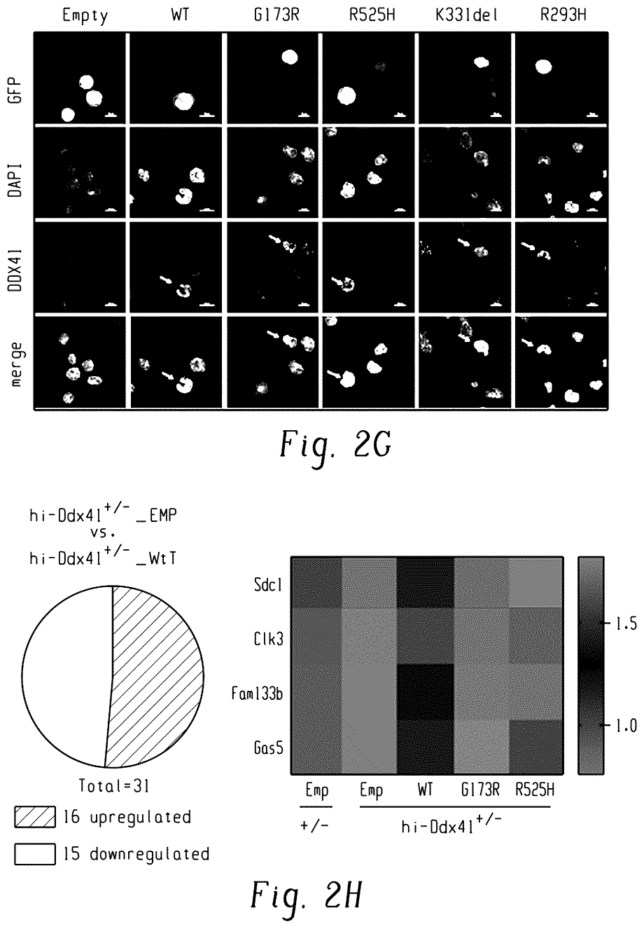

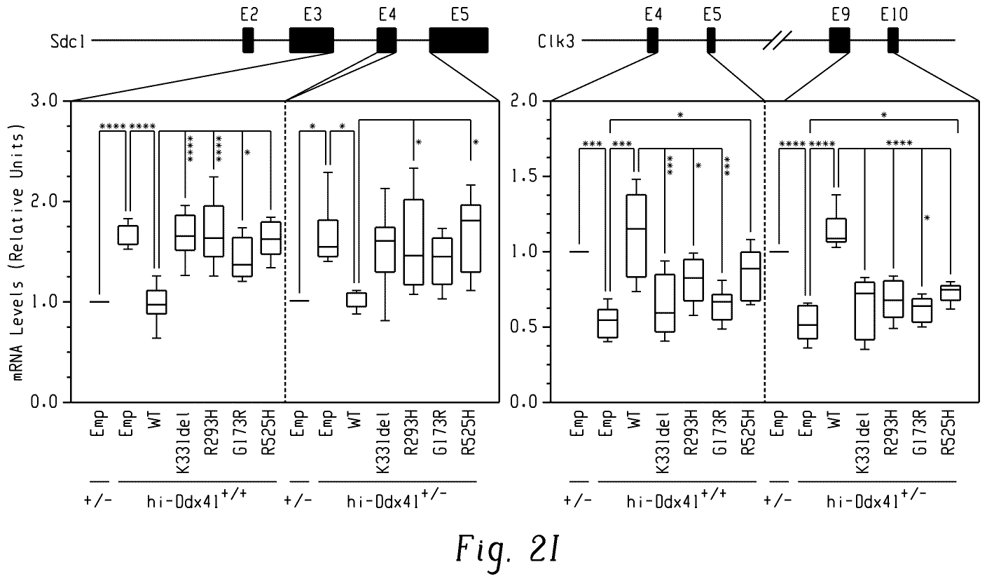

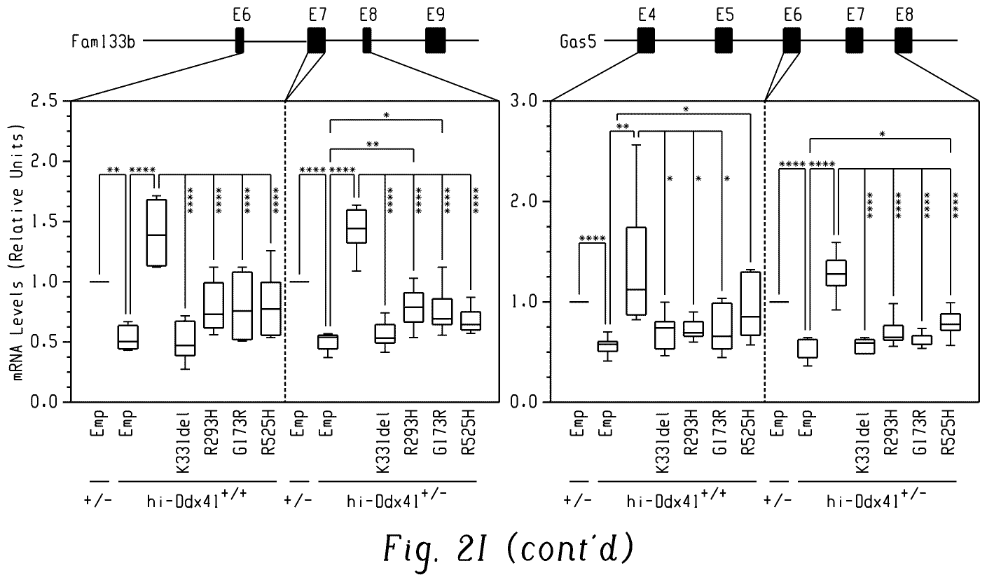

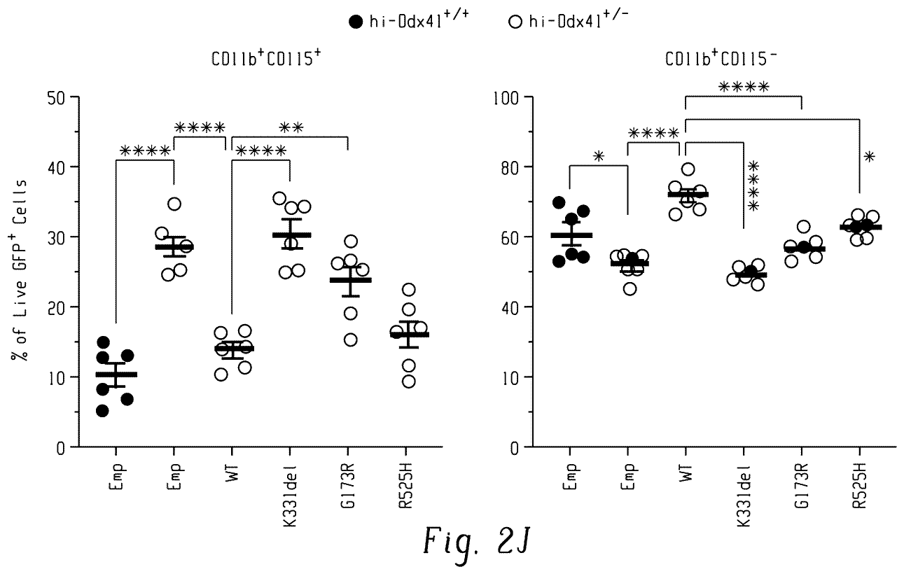

Myeloid malignancy occurring in the context of DDX41 germline variants feature a late age of onset and often acquire an additional somatic DDX41 mutation. Given these attributes and lethality in homozygous knockout mice prior studies to dissect DDX41 mechanisms have been conducted with heterozygous knockout mice, in conjunction with aging or the acquisition of a somatic mutation. As ascribing mechanistic consequences of variants in proteins that profoundly influence cellular functions can be complex, an assay was developed to enable facile mechanistic dissection and clinical curation of DDX41 variants (FIG. 2A) as a complement to complex in vivo strategies. The assay utilizes Hoxb8-immortalized mouse fetal liver progenitor cells, which exhibit a normal myeloid progenitor cell phenotype. These cells were engineered with CRISPR-Cas9 to generate Ddx41^{+/-} clonal lines with reduced endogenous DDX41 protein levels. As Ddx41-nullizygous mice are lethal, our system, in which DDX41 protein is approximately 50% lower than that of hi-Ddx41^{+/+} cells, minimizes the potential for deleterious effects on cellular functions and provides a model for human DDX41 heterozygous variants. WT or mutant DDX41 proteins can be expressed at near-physiological levels and functional consequences e.g., impact on transcript levels, quantified. The crRNA target sequences resided upstream of the exon encoding functional domains (exon 3 (A)), or within exons encoding the N-terminal DEAD box domain (exon 6 (B) and exon 8 (C), FIG. 2B). The Ddx41+/− cells were validated by Sanger sequencing of genomic DNA at and surrounding the target sequence (FIG. 2C). Semi-quantitative western blotting revealed reduced DDX41 levels in hi-Ddx41^{+/-} relative to hi-Ddx41^{+/+} cells (FIG. 2D). hi-Ddx41^{+/+} and hi-Ddx41^{+/-} cells were infected with retrovirus expressing GFP alone (Empty) or GFP with DDX41 or myeloid malignancy associated DDX41 variants. Because DDX41 protein sequence is highly conserved (99%) between human and mice, human DDX41 was utilized for genetic rescue. After two days, GFP-positive cells were sorted and analyzed by western blotting (FIG. 2E, F). Importantly, the proteins were not overexpressed, as the aggregate level of DDX41 and the DDX41 variant in hi-Ddx41^{+/-} cells resembled that of hi-Ddx41^{+/+} cells (FIG. 2F). While an alternative strategy to analyze DDX41 variants would involve variant knock-in at the endogenous DDX41 locus, this assay allows for multiple variants to be analyzed in the same cellular environment without potential complications from differential genetic influences arising from the use of multiple knock-in lines.

DDX41 has three putative NLS sequences and localizes to the nucleus in the steady-state. All disease-associated variants tested in the genetic-rescue assay were nuclear-localized, resembling DDX41. Thus, the sequence variation did not overtly alter subcellular localization (FIG. 2G). In addition, the cell cycle status of hi-Ddx41^{+/+} and hi-Ddx41^{+/-} cells expressing DDX41 or variants was indistinguishable. To identify DDX41-sensitive molecular processes in hi-Ddx41+/− cells that can be leveraged to quantitatively compare DDX41 and variant activities, RNA-seq was integrated into the rescue system (FIG. 2H). DDX41 expression in hi-Ddx41^{+/-} cells significantly altered the abundance of a restricted ensemble of transcripts; 16 and 15 were upregulated and downregulated, respectively (FIG. 2H, left).

To prioritize candidates from among the DDX41-regulated transcripts, mouse specific genes and predicted genes were excluded, and only genes with TPM>1 were considered. The transcripts were quantified in hi-Ddx41^{+/-} cells, with or without DDX41 expression, to determine if DDX41 rescues transcript levels. Among these transcripts, it was determined whether they were also regulated by DDX41 disease variants, and four were analyzed further (FIG. 2H, right). Syndecan-1 (Sdc1) RNA was elevated 1.6-fold (p=0.0008) in hi-Ddx41^{+/-} versus hi-Ddx41^{+/+} cells, and DDX41, but not the G173R and R525H variants, decreased its expression (68%, p=0.004). Fam133b, Clk3, and Gas5 mRNA levels were lower in hi-Ddx41^{+/-} versus hi-Ddx41^{+/+} cells. DDX41, but not the mutants, increased Fam133b mRNA levels (1.7-fold, p=0.0149). DDX41 increased Clk3 and Gas5 mRNA levels 1.4- and 1.6-fold (p=0.0098 and 0.0564), while the G173R variant was inactive; R525H had reduced activity (1.2-1.4-fold increase, p=0.1123 and 0.2617).

To determine if the novel variants retain competence to alter the levels of DDX41-regulated transcripts, qRT-PCR was conducted with two primer sets that amplify distinct exons of each gene (FIG. 21). Sdc1 expression was 1.7-fold higher (p<0.0001; E3-4, p=0.0194; E4-5) in hi-Ddx41^{+/-} versus hi-Ddx41^{+/+} cells, and DDX41 decreased its expression. Fam133b, Gas5, and Clk3 expression were 40-50% lower (Fam133b; p=0.0075 (E6-7), p<0.0001 (E7-8), Gas5; p<0.0001 (E4-6 and E6-8), and Clk3; p=0.0009 (E4-5), p<0.0001 (E9-10), respectively) in hi-Ddx41^{+/-} versus hi-Ddx41^{+/+} cells, and DDX41 rescued expression. Consistent with the RNA-seq data, G173R was inactive. R525H was not competent to regulate Sdc1 and Fam133b mRNAs, and its activity to regulate Clk3 and Gas5 was attenuated (Clk3, 24% (p=0.1259, E4-5) and 26% (p<0.0001, E9-10), Gas5, 30% (p=0.259, E4-6) and 39% (p<0.0001, E6-8) reduction). K331del variant did not alter the mRNA levels, and R293H variant activity was attenuated relative to DDX41 (Sdc1, 42% (p<0.0001, E3-4) and 37% (p=0.0477, E4-5), Fam133b, 44% (p=0.003, E6-7) and 45% (p=0.0094, E7-8), Clk3, 29% (p=0.0446, E4-5) and 40% (p<0.0001, E9-10), Gas5, 45% (p=0.022, E4-6) and 47% (p<0.0001, E6-8) reduction).

It was further tested whether DDX41 and variants differed with respect to their activity to regulate hematopoietic differentiation in the genetic rescue assay (FIG. 2J). At an early stage of differentiation, although the wild type and heterozygous cells were morphologically indistinguishable (data not shown), changes in cell surface markers were detectable by flow cytometry (FIG. 2J, data not shown). The immortalized progenitor cells have the potential to undergo monocytic and granulocytic differentiation. Ddx41 haploinsufficiency increased monocytic differentiation (CD11b⁺CD115⁺; 2.2-fold increase, p<0.0001). DDX41 expression in hi-Ddx41^{+/-} cells reduced the monocytic population (CD11b⁺CD115⁺; 40% relative to control retrovirus-infected hi-Ddx41^{+/-}, p<0.0001), while promoting granulocytic differentiation (CD11b⁺CD115⁻; 1.3-fold increase, p<0.0001). However, the K331del and G173R variants lacked activity. DDX41 and R525H and G610S exhibited comparable activities (CD11b⁺CD115⁺; 30% (R525H, p=0.0012) and 50% (G610S, p<0.0001) reduction relative to control retrovirus-infected hi-Ddx41^{+/-}, CD11b⁺CD115⁻; 1.2-fold (R525H, p=0.001) and 1.5-fold (G610S, p<0.0001) increase). Thus, the germline K331del and G173R variants were overtly defective, whereas G610S, a variant of uncertain significance (data not shown) retained activity, and the common somatic variant R525H retained a subset of activities. This genetic rescue-based differentiation assay complements the transcript quantification assay for curating functional attributes of human DDX41 variants.

### Example 3: Mechanism of Gene Regulation by DDX41

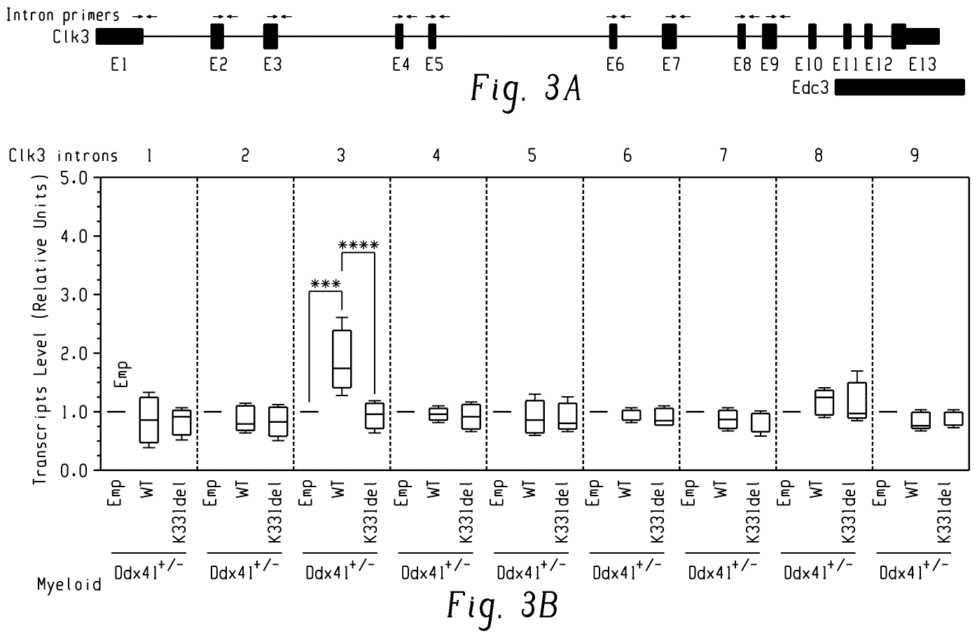

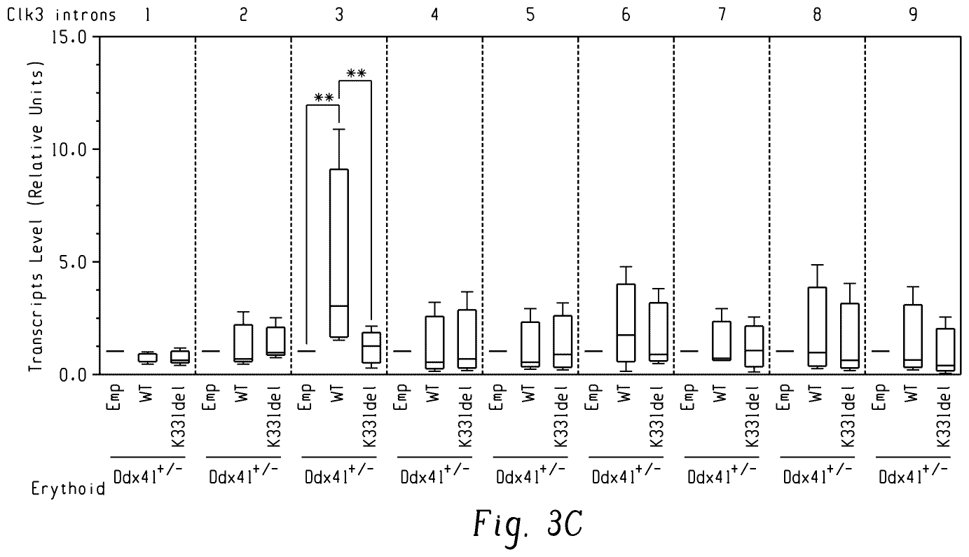

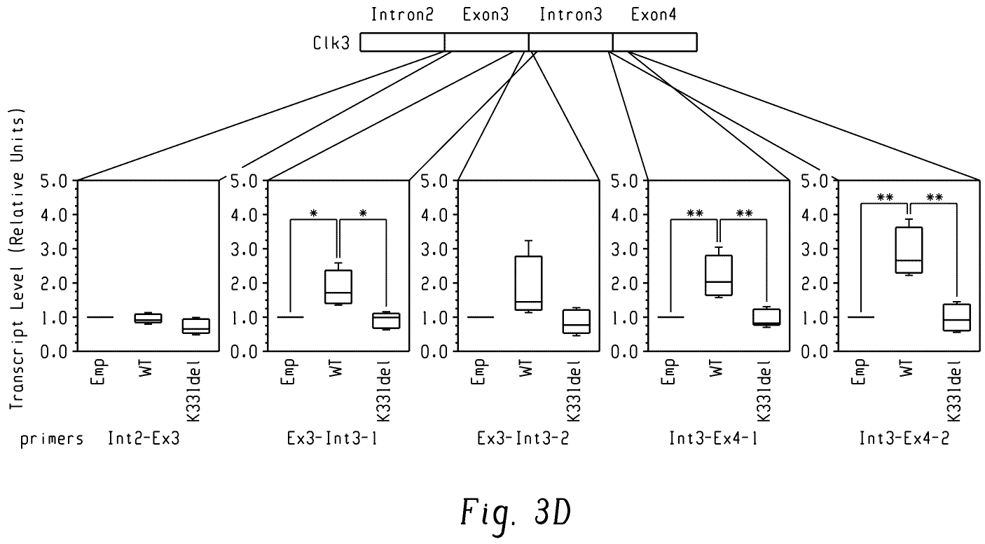

To investigate the mechanism of gene regulation by DDX41, it was first checked whether DDX41 regulates the transcription of Clk3. To measure primary transcript levels of CLK3 by RT-qPCR, primer sets were applied on adjacent exons and introns (FIG. 3A).

Primer sets for FIG. 3

| | | SEQ |
|---|---|---|
| | | ID |
| | SEQUENCE | NO: |
| Clk3-ei1-Forward | TGGGAGACGGTGAGTGT | 24 |
| Clk3-ei1-Reverse | GAAATAGGTCCAAGCCGAGAC | 25 |
| Clk3-ei2-Forward | CATCCCGAAGGGAGCCT | 26 |
| Clk3-ei2-Reverse | AGAGCTCTGGGCCATTCTA | 27 |
| Clk3-ei3-Forward | GTACCAGGTCTTGTAGCAGTG | 28 |
| Clk3-ei3-Reverse | GAGAAAGAGGTGAGGCCTTTAG | 29 |
| Clk3-ei3-2Forward | TCACAAACGCCGTACCAG | 30 |
| Clk3-ei3-2Reverse | GAGAAGCGAGAAAGAGGTGAG | 31 |
| Clk3-ie3-Forward | ACCTCAATCTGTCAATCGGAA | 32 |
| Clk3-ie3-Reverse | CTCCTTGTCATCTTCCACACTC | 33 |
| Clk3-ie3-2Forward | ATTGGTCGGATGGCGTTT | 34 |
| Clk3-ie3-2Reverse | GGCCCTCCTTGTCATCTTC | 35 |
| Clk3-ei4-Forward | TGCCGGATCGGCGATTG | 36 |
| Clk3-ei4-Reverse | GGGTAGAAAGTAGAGAGGTAAAGTT | 37 |
| Clk3-ei5-Forward | GTGGAGTGCTTGGACCAT | 38 |
| Clk3-ei5-Reverse | ACCCAACACATCCAGCAAT | 39 |
| Clk3-ei6-Forward | ATCAAGGAGAAAGACAAGGAAA | 40 |
| Clk3-ei6-Reverse | GCAGCTGGAACTAGAGAATG | 41 |
| Clk3-ei7-Forward | GCTCTGTCATGCCCTTAGAT | 42 |
| Clk3-ei7-Reverse | GGCCTGTAAGACCTTGTAGAAT | 43 |
| Clk3-ei8-Forward | CCAGAGAACATCTTGTTTGTGAAT | 44 |
| Clk3-ei8-Reverse | CTTAGAGCAGGGCCACAC | 45 |
| Clk3-ei9-Forward | ACTACCGCCCACCTGAG | 46 |
| Clk3-ei9-Reverse | TGATAACTGCCAGATGTAAACAATG | 47 |
| Clk3-intron3exon5- | ACCTCAATCTGTCAATCGGAAC | 48 |
| Forward (qPCR, | | |
| PCR) | | |
| Clk3-intron3exon5- | AGCACTCCACCACCTTG | 49 |
| Reverse (qPCR, | | |
| PCR) | | |
| Ybx1-202-Forward | TGCAGGAGAGCAAGGTAGA | 50 |
| Ybx1-202-Reverse | CTGAAATGAATGCTTCGGAATCG | 51 |
| Ythdf2-202- | TCTTCTCGGACGACTTGCT | 52 |
| Forward | | |
| Ythdf2-202- | GGGCTCAAGTAAGGTTCGAAAT | 53 |
| Reverse | | |
| Khdc4-206- | TACAGGCTTCTCCAGTCAGA | 54 |
| Forward | | |
| Khdc4-206- | TGTCCCTCTCTCGCTCTTT | 55 |
| Reverse | | |
| Ddx5-208-Forward | CCACACTGCAACACCTTACA | 56 |
| Ddx5-208-Reverse | TCCTTCATGCCTCCTCTACC | 57 |
| CLK3- | TTGGTCGGATGGCGTTTC | 58 |
| intron3_exon5- | | |
| Forward | | |
| CLK3- | TTGCCAAAGGTGCCTTCA | 59 |
| intron3_exon5- | | |
| Reverse | | |

The exon-intron junction before intron 10, which overlaps with the Edc3 gene, was analyzed by RT-qPCR. Interestingly, only the intron 3 region was increased by 1.7-fold by DDX41 (p<0.001), but not by the pathogenic variant (K331del) in hi-Ddx41^{+/-} (FIG. 3B) In addition, a distinct cellular system (G1E-ER-GATA1 pro-erythroblast, which is relevant to DDX41 actions on the erythroid system in humans) was developed that reiterates the conclusions about the merits of the genetic rescue approach to identify pathogenic variants of DDX41. Consistent with myeloid progenitor (hi-progenitor), DDX41 increased the intron 3 contained transcript by 2.5-fold (p<0.01), whereas K331del did not have an effect (FIG. 3C) Moreover, an intron 3-specific increase was confirmed using two sets of additional primers on the opposite end of intron (3′ of intron 3 and 5′ of exon 4; 2-(primer set 1, p<0.01) and 2.7-(primer set 2, p<0.01) fold (FIG. 3D). These data indicate that DDX41, but not the pathogenic variant (K331del), controls cellular transcript levels by promoting the inclusion of select introns into what should be the fully processed mRNA. The intron-retained transcript is predicted to be stuck in the nucleus. In the case of an inactive variant, it does not do this, and these accumulated transcripts are likely to serve as a reservoir for subsequent use when the cell has a higher demand for protein production.

In conclusion, a genetic rescue assay was developed that quantitatively discriminated activities of DDX41 and myeloid malignancy-associated DDX41 genetic variants. The analyses described herein revealed that the variants were impaired in their intrinsic RNA regulatory activities and to induce monocytic differentiation markers.

### Example 4: DDX41-Dependent Splicing Regulates the Splicing Factor Kinase CLK3

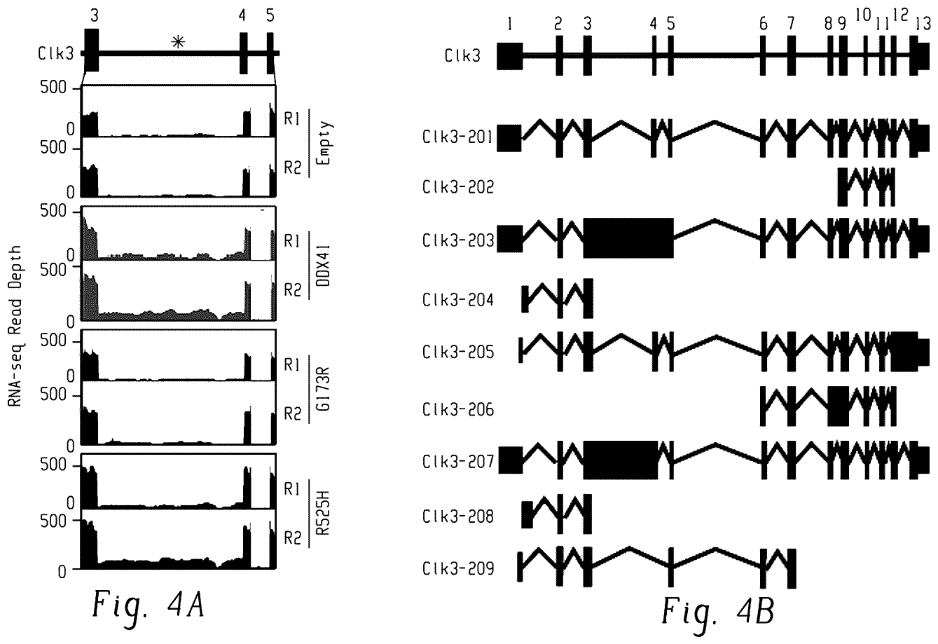

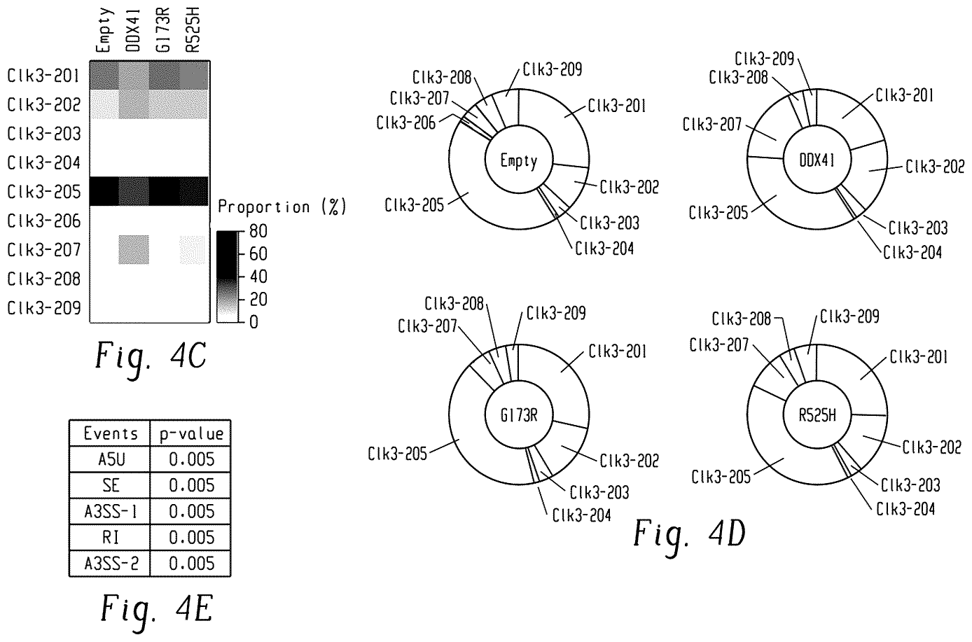

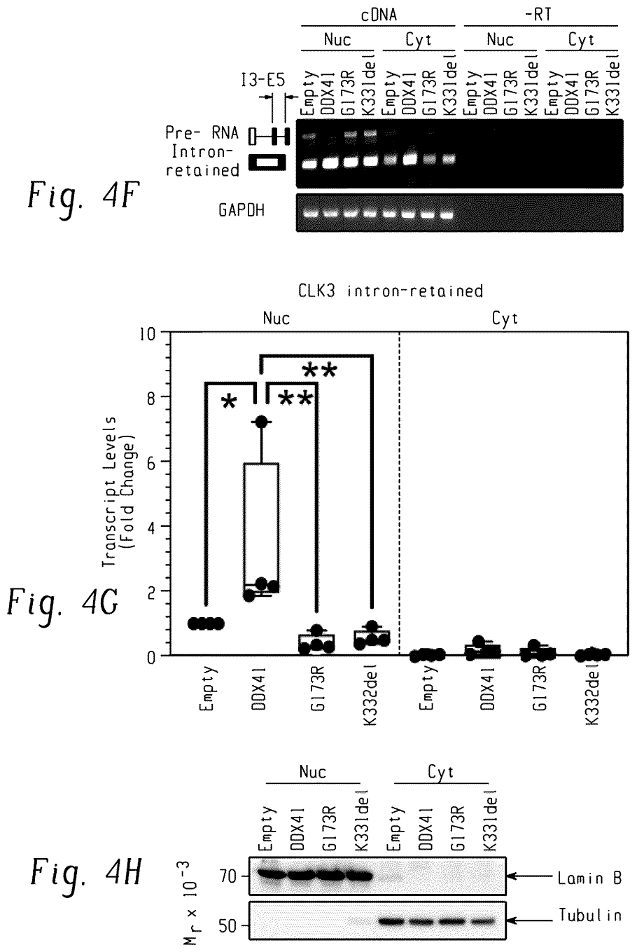

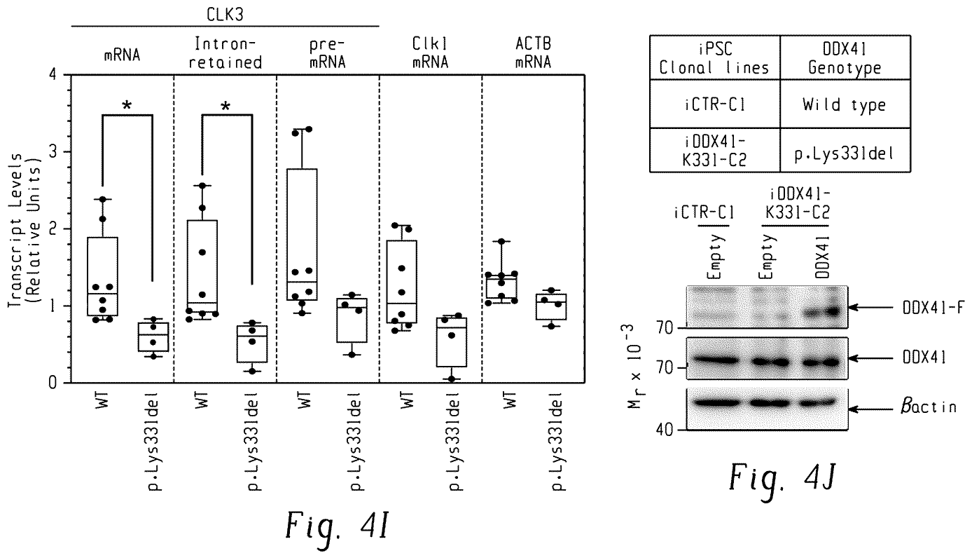

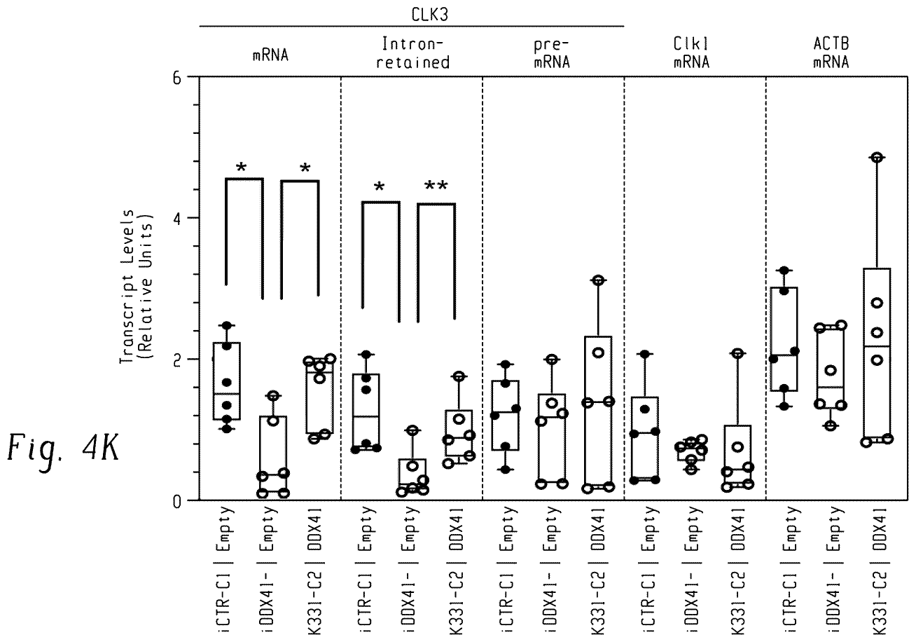

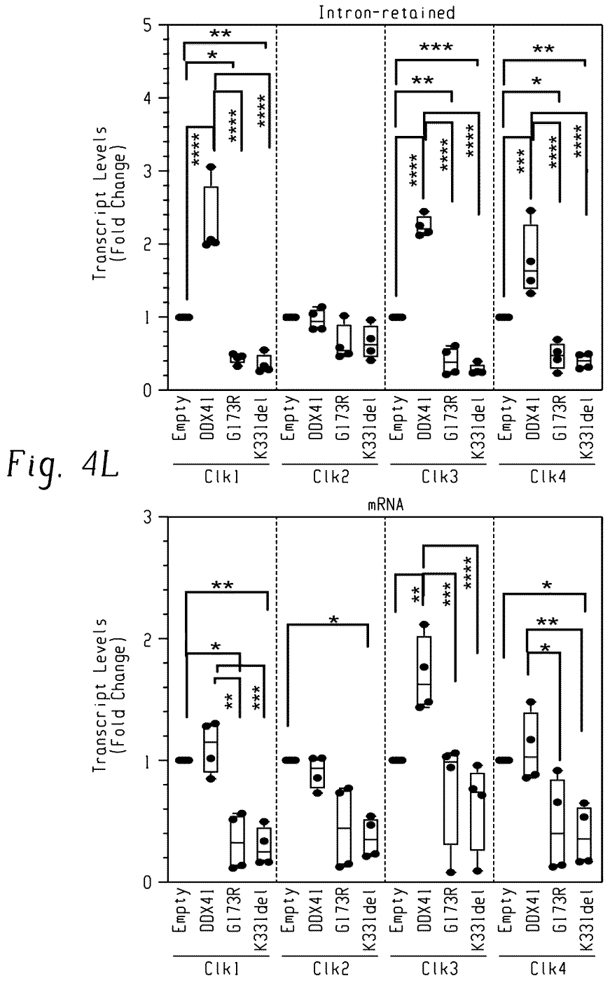

DDX41 regulates the levels of 31 mRNAs in the rescue system. Comparison of differentially regulated transcripts with differentially expressed genes (DEGs) (data not shown) revealed seven DDX41-regulated genes (Cc2d1b, Chchd1, Clk3, Fam133b, Gm47283, Prpf18, and Slc25a28) at transcript isoform and mRNA levels. G173R and R525H failed to regulate these mRNAs and their isoforms. For example, DDX41 increased the contribution of Clk3 intron 3 to transcripts, R525H was less active, and G173R lacked activity (FIG. 4A). Since mouse cells harbor nine Clk3 isoforms (FIG. 4B), we used TPMs to calculate the ratio of each isoform to total transcript (FIG. 4C and 4D). DDX41 and R525H increased intron 3-retained transcript (Clk3-207) 4.6- and 2.6-fold, respectively; G173R lacked activity. DDX41 and variants did not alter intron 3/intron 4-retained transcript (Clk3-203). DDX41 regulated Clk3-207 alternative 5′ UTR, 3′ splicing site, exon skipping, and intron retention (FIG. 4E). By regulating alternative splicing, DDX41 changed the proportion of a particular Clk3 transcript isoform.

As intron retention increases the probability of transcript accumulation in the nucleus, we analyzed transcript subcellular distribution in the rescue system (FIG. 4F, 4G, and 4H). Clk3-207 was predominantly nuclear (FIG. 4F, 4G). DDX41 increased intron retention 2-fold (p<0.05); G173R lacked activity as did K331del, another pathogenic germline variant.

To establish the relationship of DDX41-regulated Clk3 transcript isoforms in the murine system to humans, we compared primary human skin fibroblasts from a patient carrying a germline pathogenic variant (K331del) with those from an age- and gender-matched control (FIG. 41). CLK3 mRNA was 54% lower in K331del versus. WT fibroblasts (p=0.0435). CLK3 intron 3 retention was 61% lower in K331del fibroblasts (p=0.0387). CLK3 pre-mRNA was also lower in K331del, but not significant. CLK1 mRNA, encoding another CLK family member, was not significantly different in the mutant cells; ACTB (FIG. 41) mRNA was constant.

To establish if DDX41 regulates CLK3 transcripts in another human system, we generated induced-pluripotent stem cells (iPSCs) from skin fibroblasts from a patient with a heterozygous germline DDX41 pathogenic variant (K331del) and a DDX41-wild-type age-matched control and subjected them to feeder-free culture (FIG. 4J upper). We innovated a rescue system using lentiviral infection-based DDX41 expression. The endogenous DDX41 level of WT and mutant iPSCs was comparable (FIG. 4J lower). The pathogenic consequences of K331del are therefore unrelated to altering the steady-state protein level. CLK3 intron-retained transcript levels were 33% lower (p<0.05) in mutant vs. WT iPSCs, and expressing DDX41 increased the transcript (p<0.01; FIG. 4K). CLK3 mRNA was 33% lower in mutant vs. WT iPSCs (p<0.05) and rescued by DDX41 (p<0.05). CLK3 pre-mRNA levels were similar in WT and mutant iPSCs and unaffected by the DDX41 variant. CLK1 and ACTB mRNAs were unchanged between WT and mutant iPSCs, suggesting DDX41 regulates CLK3 alternative splicing but not transcription. In murine myeloid progenitors, primary human skin fibroblasts, and human iPSCs, DDX41 regulated Clk3 transcript isoforms.

Since four murine and human Cdc2-like kinase (Clk) family members exist, we asked if DDX41 regulates other Clk transcripts in mouse myeloid progenitors (FIG. 4L). DDX41 increased Clk1 and Clk4 intron-retained transcripts 2.0-(p<0.0001) and 1.7-fold (p<0.001), respectively (FIG. 4L upper). DDX41 variants decreased transcript levels (Clk1, 54% by G173R (p-value=0.019); 63% by K331del (p-value=0.004); Clk4, 53% by G173R (p-value=0.035); 60% by K331del (p-value=0.009), respectively). Unlike Clk3 mRNA, DDX41 did not increase Clk1 and Clk4 mRNAs (FIG. 4L lower).

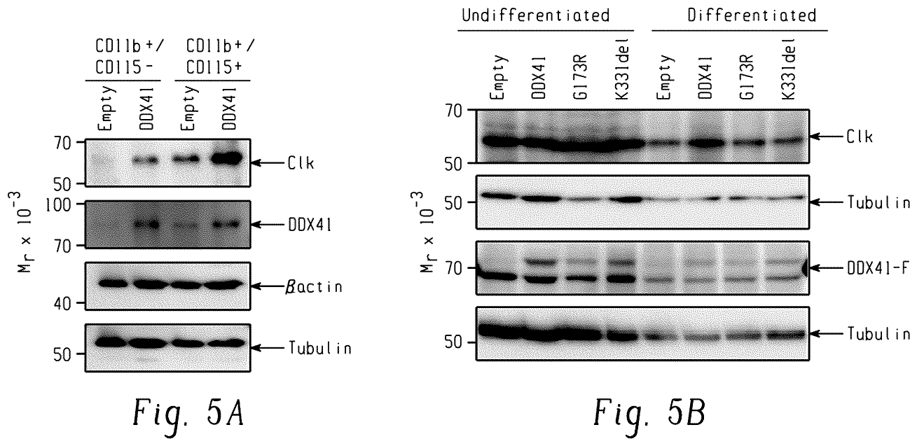

Since DDX41 regulated monocytic and granulocytic differentiation of myeloid progenitors, we analyzed the intron-retained Clk3 transcript in CD11b⁺/CD115⁻ (granulocytic) or CD11b⁺/CD115⁺ (monocytic) Ddx41^{+/-} and genetically rescued cells by RT-qPCR (data not shown) and quantified CLK3 protein (FIG.5A). DDX41 increased intron-retained transcripts in granulocytic and monocytic populations (data not shown) and increased CLK3 protein during granulocytic and monocytic differentiation. Comparison of DDX41 and variant activities to alter CLK3 protein in the rescue assay with undifferentiated and differentiated cells revealed that DDX41 and variants did not affect CLK3 in undifferentiated cells. However, CLK3 decreased upon differentiation, and DDX41, but not G173R and K331del, elevated CLK3 in this context (FIG. 5B). DDX41 regulation of Clk3 transcripts therefore controls CLK3 abundance during myeloid differentiation.

### Example 5: CLK3-dependent and -independent Mechanisms of DDX41-regulated Alternative Splicing

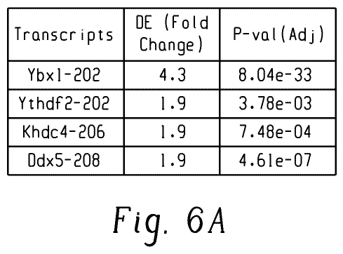

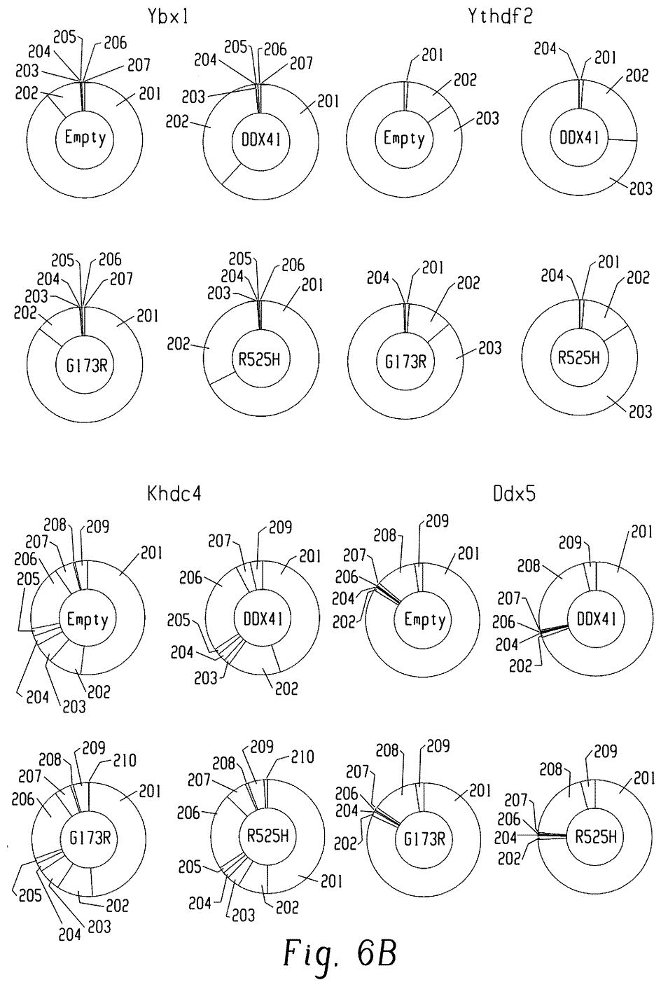

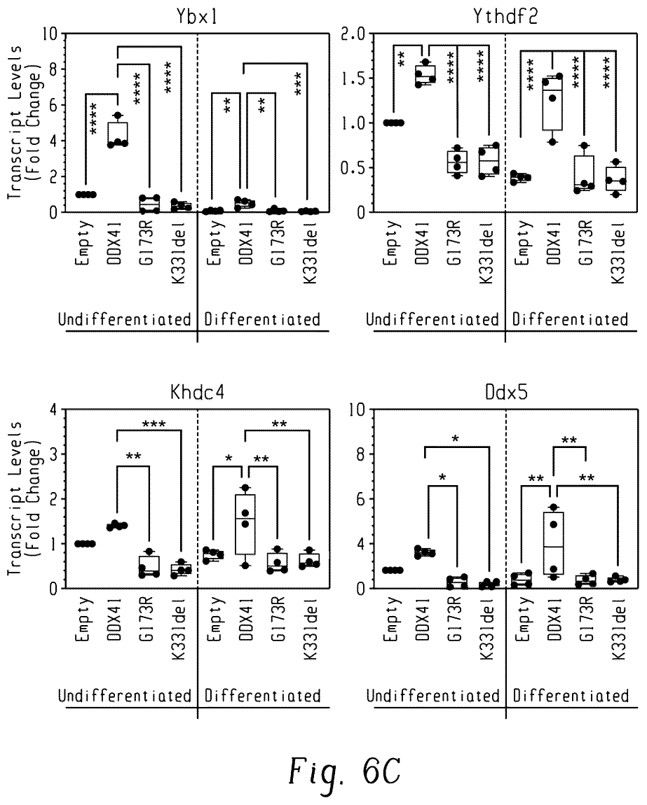

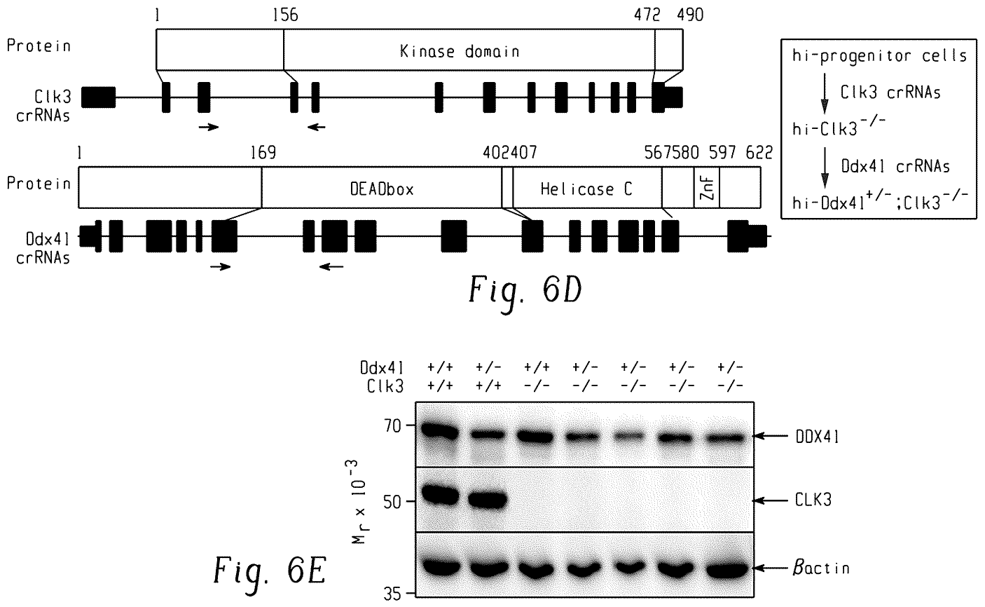

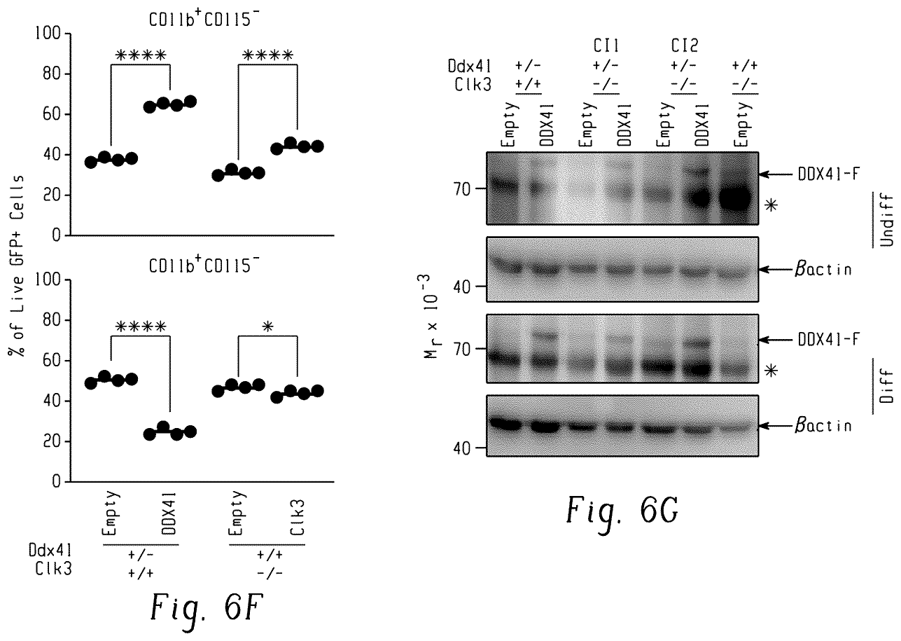

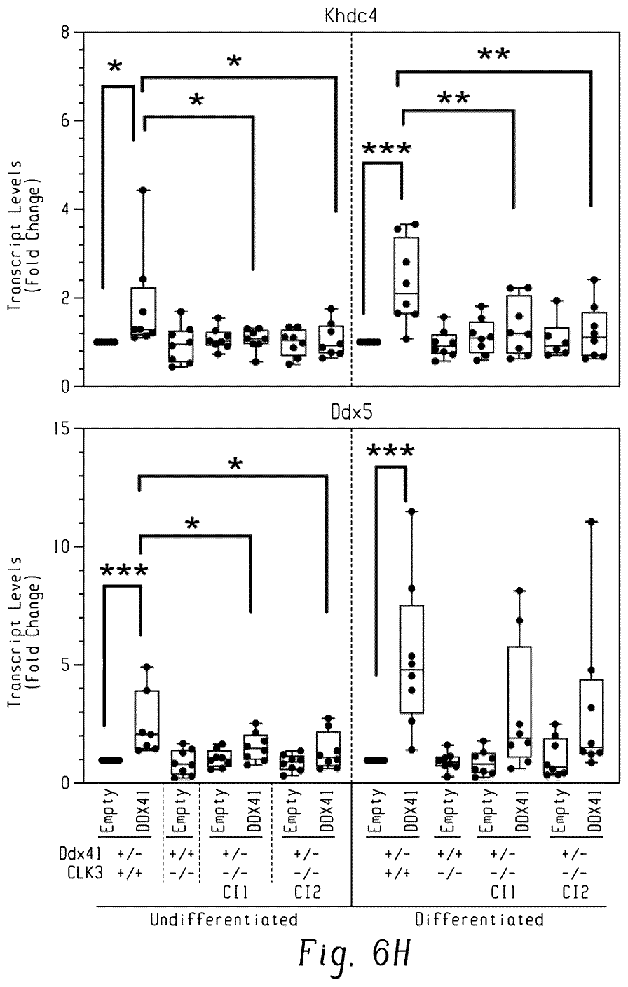

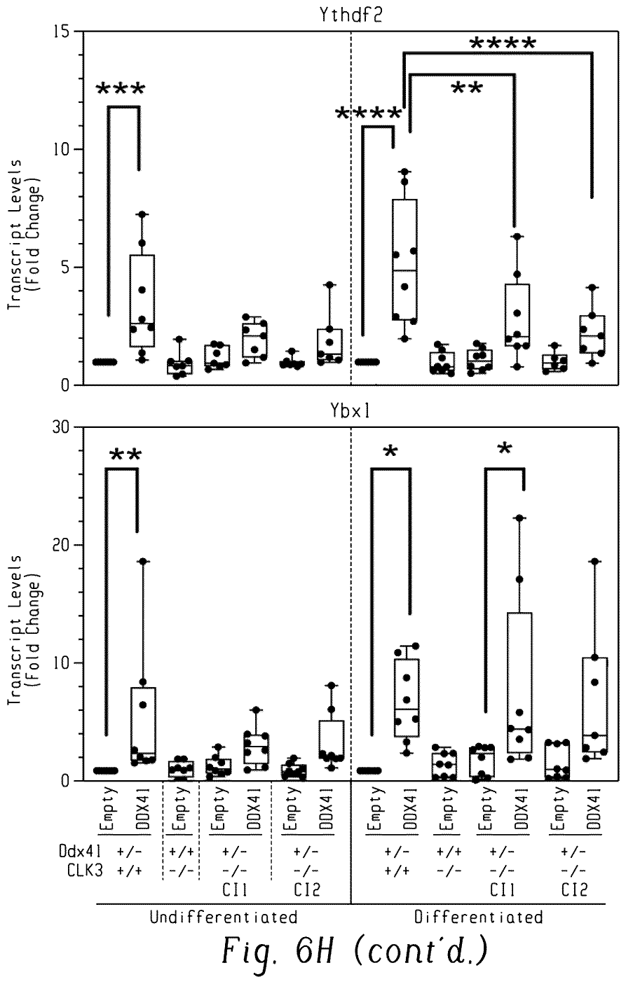

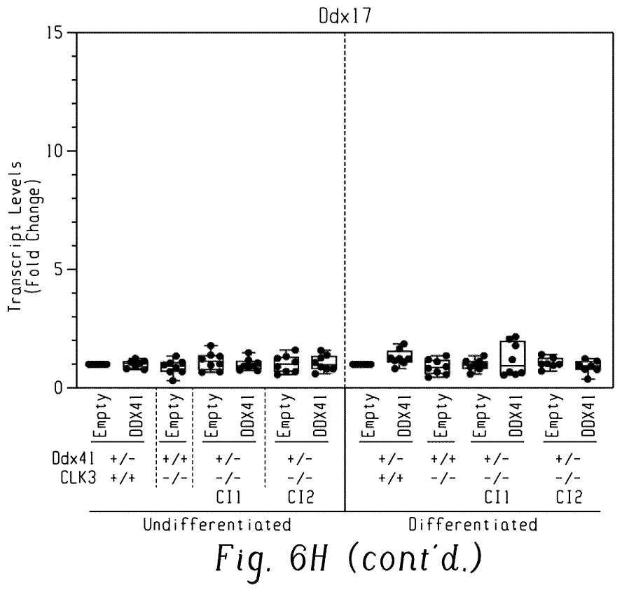

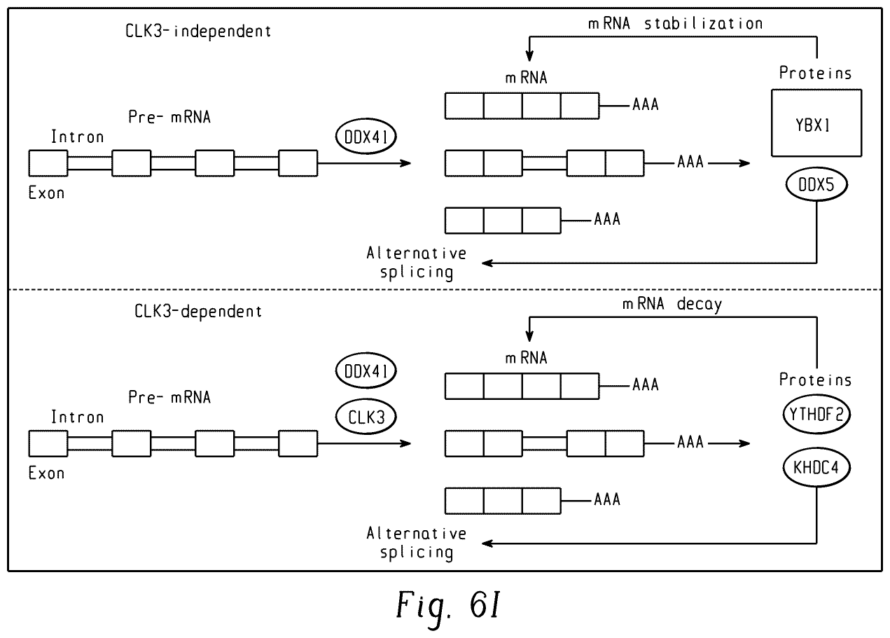

Since CLK3 induces SR protein phosphorylation and regulates their activities, it is attractive to consider whether CLK3 operates in the DDX41 mechanism to control alternative splicing. To test whether CLK3 mediates DDX41 functions, we identified 515 DDX41-regulated transcripts in DTE analysis (adjusted p-value<0.1, |log2(FoldChange)|>0.5) that were not regulated by pathogenic variants. Four transcripts (Ybx1-202, Ythdf2-202, Khdc4-206, and Ddx5-208) were analyzed to test if CLK3 mediates DDX41-dependent splicing (FIG. 6A). The proportion of each transcript was calculated using TPM values (FIG. 6B). DDX41 increased the proportion of Ybx1-202 3.7-fold (9.8% in control and 37% in DDX41). R525H increased Ybx1-202 3.2-fold, while G173R was inactive. DDX41 increased Ythdf2-202 proportion 1.8-fold (14 to 25%); G173R and R525H were inactive. DDX41 increased Khdc4-206 proportion 1.4-fold (18 to 27%); G173R and R525H were inactive. DDX41 increased Ddx5-208 proportion 2.1-fold (12 to 25%). G173R lacked activity, while R525H activity was attenuated (1.7-fold). DDX41 regulation of these transcripts was confirmed by RT-qPCR (FIG. 6C). Analysis of transcript levels in hi-Ddx41^{+/-} cells expressing G173R and K331del in undifferentiated and differentiated states confirmed that DDX41, but not the variants, increased Ybx1-202 in undifferentiated (4.3-fold; p-value<0.0001) and differentiated cells (5.4-fold; p-value=0.0013). DDX41 increased Ythdf2-202 (1.5-fold, p-value=0.0023 in undifferentiated; 3-fold, p-value<0.001 in differentiated); variants were inactive. DDX41 increased Khdc4-206 and Ddx5-208 (Khdc4-206; 1.33-fold, p-value=0.4835 (undifferentiated), 1.8-fold, p-value=0.0218 (differentiated), Ddx5-208; 2-fold, p-value=0.4322 (undifferentiated), 5-fold, p-value=0.0061 (differentiated)); variants were inactive in both conditions.

To test if CLK3 mediates DDX41 function, we generated double-mutant Clk3^{-/-}; Ddx41^{+/-}clonal lines from HoxB8-immortalized cells using CRISPR/Cas9 (FIG. 6D). hi-Clk3^{-/-} cell lines were engineered with crRNAs targeting genomic sequences upstream of the CLK3 kinase domain. Two hi-Clk3^{-/-} lines were edited with crRNAs targeting upstream of the DDX41 DEAD box. Consistent with DDX41 being essential for viability, only Ddx41^{+/-} lines were obtained. DDX41 and CLK3 were analyzed by semi-quantitative Western blotting (FIG. 6E). The granulocytic (CD11b⁺CD115⁻, ˜40% of live GFP⁺cells) and monocytic population (CD11b⁺CD115⁺, ˜45% of live GFP⁺cells) of GFP-expressing Clk3^{-/-} cells resembled that of GFP-expressing Ddx41^{+/-}. CLK3 loss did not affect monocytic differentiation (CD11b⁺CD115⁺; FIG. 6F, lower), and granulocytic differentiation increased slightly when CLK3 was expressed in the hi-Clk3^{-/-} lines (FIG. 6F, upper).

We innovated a rescue system with Clk3^{-/-};Ddx41^{+/-} clonal lines without or with myeloid differentiation. Lentivirus-expressed DDX41 was measured with anti-Flag antibody (FIG. 6G). DDX41 increased CD11b⁺CD115⁻(granulocytic) and decreased CD11b⁺CD115⁺ (monocytic) populations in Ddx41^{+/-} and Clk3^{-/-};Ddx41^{+/-} lines (data not shown). DDX41 increased Ybx1-202 in Ddx41^{+/-} undifferentiated cells (5.5-fold, p-value=0.0041) (FIG. 6H). The rescue decreased by 43% and 40% in the Clk3^{-/-}; Ddx41^{+/-} lines (clone1 (Cl1); 3.1-fold; p-value=0.2929, clone2 (Cl 2); 3.3-fold; p-value=0.3708, respectively) vs. Ddx41^{+/-} control. Upon differentiation, DDX41 rescue was detected in Clk3^{-/-};Ddx41^{+/-} clones (Ddx41^{+/-}; 6.8-fold; p-value=0.022, Cl1; 7.7-fold; p-value=0.0213, and Cl2; 14-fold; p-value=0.563). DDX41 increased Ythdf2-202 in Ddx41^{+/-} cells (2-fold, p-value=0.0012), and rescue decreased by 43% (p-value=0.0894) and 45% (p-value=0.0606) in undifferentiated Clk3^{-/-};Ddx41^{+/-} cells. Upon differentiation of Ddx41^{+/-} cells, DDX41 increased Ythdf2-202 5.1-fold (p-value<0.0001), and rescue decreased by 45% (Cl1; p-value=0.0134) or 56% (Cl2; p-value=0.0016) in Clk3^{-/-};Ddx41^{+/-} cells. DDX41 increased Khdc4-206 1.8-fold in undifferentiated (p-value=0.0119) and 2.3-fold in differentiated (p-value=0.001) Ddx41^{+/-} cells, but did not rescue the transcript in undifferentiated or differentiated Clk3^{-/-};Ddx41^{+/-} cells. DDX41 increased Ddx5-208 2.5-fold (p=0.0003) and was 38% (Cl1: 1.3-fold; p=0.0423) and 45% (Cl2: 2.3-fold; p=0.0116) lower in undifferentiated Clk3^{-/-}; Ddx41^{+/-} cells. DDX41 increased Ddx5-208 in Ddx41^{+/-} cells upon differentiation, and rescue decreased in Clk3^{-/-};Ddx41^{+/-} lines (DDX41; 5.3-fold (p=0.0007); Cl1; 3.2-fold (p=0.2029); Cl2; 4.2-fold (p=0.2329)). DDX41 did not alter Ddx17 mRNA encoding an unrelated DEAD-box helicase. These results indicate that DDX41 utilizes CLK3-dependent and CLK3-independent mechanisms to regulate transcript isoforms.

By generating an average of seven mRNA isoforms per human gene., alternative splicing greatly expands information content encoded by the genome. Maintaining the integrity of the splicing machinery is therefore crucial, and pathogenic mutations in splicing components are associated with, and in some cases cause, oncogenesis. A characteristic example of this scenario involves the splicing regulator DDX41, as pathogenic germline genetic variation creates a predisposition to bone marrow failure and myeloid malignancy. Herein, we utilized global and molecular strategies to establish the repertoire of DDX41-regulated transcripts in a progenitor cell. DDX41 pathogenic genetic variants were severely compromised in controlling multiple steps in splicing. In the context of a pathogenic germline genetic variant that creates an MDS/AML predisposition, the major somatic variant R525H often emerges concomitant with progression to myeloid malignancy. At the global level, R525H exhibited significant splicing defects, yet the severity of the defects was considerably less than for germline variants analyzed.

Splicing, like diverse nucleic acid transactions, requires ensembles of factors with interlinked mechanisms. Given the links between splicing mechanisms and phenotypic diversification, and the requirement for high integrity of splicing mechanisms, it is attractive to consider mechanisms in which a splicing regulator subject to dysregulation by germline and somatic genetic variation controls splicing of transcripts encoding additional splicing regulators. This mechanism is exemplified by DDX41 regulating the splicing factor kinase CLK3. To evaluate functional ramifications, we innovated Clk3^{-/-} progenitors in the context of DDX41 heterozygosity, which enabled a rescue system to analyze DDX41 mechanisms without CLK3. We analyzed splicing with Ybx1-202, Khdc4-206, Ythdf2-202, and Ddx5-208 (FIG. 6). Ybx1 encodes a Y-box transcription factor that stabilizes mRNAs by binding to eIF4E⁵¹ and stabilizes m6A-tagged RNAs, such as BCL2, by cooperating with IGF2BP in myeloid leukemia cells. Khdc4 encodes a K homology domain protein involved in alternative splicing that positions the CDC5L-SNEV complex at the splice sites. Ythdf2 encodes a YTH domain family protein and regulates mRNA decay by altering mRNA localization from ribosome to processing bodies. Ddx5 encodes a DEAD-box helicase family member functioning as a transcriptional coactivator of p53-responsive promoter genes in cancer cell lines and may regulate alternative splicing of pre-mRNA in prostate cancer cells. We demonstrated that DDX41-mediated regulation of these transcripts decreased in CLK3-deficient progenitors, and upon differentiation, DDX41 did not increase Ythdf2 and Khdc4 transcripts without CLK3. These results support a model in which DDX41-dependent RNA regulation requires CLK3-dependent and independent pathways.

This study was conducted predominantly in genetically engineered murine Ddx41 and Clk3 double-mutant cells. Though DDX41 is a highly conserved protein, in principle, its mechanisms might at partially differ between species. To develop insights into the human relevance of the DDX41-CLK3 relationship, we conducted analyses in primary skin fibroblasts and iPSCs, which support the relevance of this relationship. Expanding mechanistic and biological studies to diverse systems will almost certainly increase the depth of the analyses described herein.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. An in vitro genetic rescue assay for identifying functionally defective DDX41 variants, comprising
identifying a DEAD-Box Helicase 41 (DDX41) variant of uncertain significance (VUS),
infecting a first Ddx41^{+/-} cell with a retrovirus expressing the DDX41-VUS,
infecting a second Ddx41^{+/-} cell with a retrovirus expressing a wild type control DDX41,
growing the first and second infected cells in culture for a period of time and quantitating mRNA expression of a DDX41-regulated transcript in both the first and second infected cells after the period of time, wherein the DDX41 related transcript comprises Sdc1, Fam133b, Gas5, Clk3, or a combination thereof,
calculating a differential expression of the DDX41-regulated transcript for the first infected cell compared the second infected cell, and
identifying the DDX-41-VUS as the functionally defective DDX41 variant wherein a change in the differential expression is 1.5-fold or greater,
wherein a Ddx41^{+/-} cell has a DDX41 expression level that is reduced by at least 50% compared to that of a Ddx41^{+/+} cell.

2. The method of claim 1, wherein identifying the DDX-41 VUS comprises identifying the DDX-41 VUS in a patient presenting with cytopenia, bone marrow hypocellularity, erythroid dysplasia, and/or myeloid malignancy, but not diagnosed with acute myeloid leukemia; or identifying the DDX-41 VUS in a database.

3. The method of claim 1, wherein quantitating mRNA expression of a DDX41-regulated transcript comprises reverse-transcriptase PCR.

4. The method of claim 1, further comprising
infecting a third Ddx41^{+/-} cell with a retrovirus expressing a functionally defective variant control DDX41,
growing the third infected cells in culture for a period of time and quantitating mRNA expression of DDX41-regulated genes in the third infected cells after the period of time, wherein the DDX41-regulated genes comprises Sdc1, Fam133b, Gas5, Clk3, or a combination thereof, and
calculating a differential expression of the DDX41-regulated genes for the first infected cell compared to the third infected cell to determine a functionally defective control.

5. The method of claim 1, further comprising
performing flow cytometry on the first infected cells and second infected cells and quantitating a monocytic marker of differentiation of hematopoietic progenitor cells and a granulocytic marker of differentiation of hematopoietic progenitor cells with marker-specific antibodies, wherein a 50% or greater increase or decrease in the quantitative level of the monocytic marker, the granulocytic maker, or both, in the first infected cells compared to the second infected cells identifies the functionally defective DDX41 variant as a pathogenic DDX41 variant.

6. The method of claim 5, wherein the monocytic marker is CD11b⁺CD115⁺ and the granulocytic marker is CD11b⁺CD115⁻.

7. The method of claim 1, wherein Ddx41^{+/-} cells were prepared from Hoxb8-immortalized mouse fetal liver progenitor cells.

8. A method of monitoring a patient for the development/progression of a myeloid malignancy, comprising
determining the presence or absence of a functionally defective DDX41 variant determined according to the method of claim 1 in a sample from the patient,
optionally determining the presence or absence of the functionally defective DDX41 variant in one or more family members of the patient, and
monitoring the patient and optionally the one or more family members for the development of one or more symptoms of myeloid malignancy when the patient and the one or more family members carry the functionally defective DDX41 variant.

9. The method of claim 8, wherein the sample is blood, bone marrow, fibroblasts, or fractional bone marrow hematopoietic stem/progenitor cells.

10. The method of claim 8, wherein the myeloid malignancy is myelodysplastic syndromes (MDS) or acute myeloid leukemia (AML).

11. The method of claim 8, wherein the one or more symptoms of myeloid malignancy comprises fever, fatigue, irregular heartbeat, dizziness, bone pain, frequent nosebleeds, bleeding and swollen gums, bruising on skin, loss of appetite, excessive sweating, shortness of breath, unexplained weight loss, headaches, diarrhea, menorrhagia, slurred speech, confusion, abdominal swelling, pale skin, seizures, vomiting, loss of balance, facial numbness, blurred vision, or a combination thereof.

12. The method of claim 8, wherein when the patient carries the functionally defective DDX41 variant, administering a treatment for the myeloid malignancy.

13. The method of claim 12, wherein the treatment is chemotherapy or a stem cell transplant.

14. The method of claim 8, wherein the subject has a risk factor for myeloid malignancy, the risk factor comprising being a smoker, being over 65 years of age, exposure to chemotherapy or radiation such as radiation therapy, having a history of a blood disorder, a family history of myeloid malignancy, or a combination thereof.

15. The method of claim 8, wherein the functionally defective DDX41 variant is Lys331del.

16. A method of identifying a patient as at risk for the development/progression of a myeloid malignancy, comprising
identifying a functionally defective DDX41 variant in the germline of the patient, wherein the functionally defective DDX41 variant is Lys331del.

17. The method of claim 16, further comprising administering a treatment for the myeloid malignancy.

18. The method of claim 17, wherein the treatment is chemotherapy or a stem cell transplant.
